(19) United States
(12) Patent Application Publication
(10) Pub. No.: US 2025/0281449 A1
(43) Pub. Date: Sep. 11, 2025

(54) NITROGEN-CONTAINING ANALOGS OF SALINOMYCIN FOR USE IN MULTIPLE MYELOMA (MM)

(71) Applicants: Centre National de la Recherche Scientifique, PARIS (FR); Centre Hospitalier Universitaire de Montpellier, MONTPELLIER (FR); Institut National de la Santé et de la Recherche Médicale, PARIS (FR); Institut Curie, Paris (FR); Université de Montpellier, MONTPELLIER (FR)

(72) Inventors: Raphaël RODRIGUEZ, PARIS (FR); Jérôme MOREAUX, MONTPELLIER (FR); Caroline BRET, MONTPELLIER (FR); Julie DEVIN, MONTPELLIER (FR); Tatiana CANEQUE COBO, PARIS (FR)

(21) Appl. No.: 18/559,022

(22) PCT Filed: May 5, 2022
(86) PCT No.: PCT/EP2022/062202

§ 371 Date: Nov. 3, 2023

(30) Foreign Application Priority Data
May 5, 2021 (EP) 21305583.3

(51) Int. Cl. A61K 31/351; A61K 31/198; A61K 31/4162; A61K 31/4433; A61K 31/454; A61K 38/07; A61P 35/00; C12Q 1/6886; G01N 33/574

(52) U.S. Cl. CPC A61K 31/351; A61K 31/198; A61K 31/4162; A61K 31/4433; A61K 31/454; A61K 38/07; A61P 35/00; C12Q 1/6886; G01N 33/57407; C12Q 2600/106; C12Q 2600/158

(57) ABSTRACT

The invention relates compound of formula (I), enantiomers, mixture of enantiomers, diastereoisomers and mixture of diastereoisomers thereof:

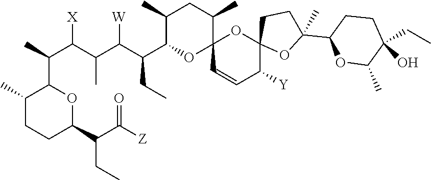

wherein W, X, Y and Z are as defined, for use in the treatment of Multiple Myeloma (MM). A pharmaceutical composition including a pharmaceutical acceptable vehicle and at least a compound of formula (I) is also included.

### CROSS-REFERENCE TO RELATED APPLICATIONS

The present application is a filing under 35 U.S.C. 371 as the National Stage of International Application No. PCT/EP2022/062202, filed May 5, 2022, entitled “NITROGEN-CONTAINING ANALOGS OF SALINOMYCIN FOR USE IN MULTIPLE MYELOMA (MM),” which claims priority to European Application No. 21305583.3 filed with the European Patent Office on May 5, 2021, both of which are incorporated herein by reference in their entirety for all purposes.

### FIELD OF THE INVENTION

The present invention relates to the field of treatment of subject affected by Multiple Myeloma (MM), as well as associated therapeutic uses and methods.

### BACKGROUND OF THE INVENTION

Multiple Myeloma (MM) account for approximately 10% of hematological malignancies and is the second most common hematological disorder. Active research on MM allowed great improvement in treatment, including proteasomes inhibitors, immunomodulatory agents or monoclonal antibodies that enhanced significantly the patient's median survival from 3-4 years in the 90's to 7-8 years nowadays.

However, there is a vital need for additional therapies since until today, MM is a condition that cannot be cured, and all patients finally relapse. Iron is essential for many fundamental cellular functions, including proliferation and DNA synthesis. The inventors demonstrated for the first time, that the iron metabolism pathway is significantly deregulated in MM subjects and could be exploited to develop novel therapeutic strategies using iron metabolism inhibitors. The inventors also built a GEP-based iron score that allow to identify MM patient with a poor outcome and dysregulation of iron metabolism that could benefit from targeted therapy. The data illustrated in the present invention demonstrated that a subgroup of high-risk MM patients could be identified with the iron-score and could benefit from Iron metabolism inhibitor and especially Ironomycin or AM23. Furthermore, combination of Ironomycin with Melphalan or immunomodulatory agents such as Lenalidomide and Pomalidomide in the present invention demonstrated a synergistic effect in MM patients.

### SUMMARY OF THE INVENTION

So a first object of the present invention is a compound of formula (I), enantiomers, mixture of enantiomers, diastereoisomers and mixture of diastereoisomers thereof:

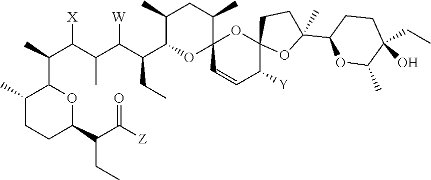

wherein:
—W is selected from the group consisting of ═O; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈;
—X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
—Y is selected from the group consisting of —OH; —N—OH; —NR₁R₂; —NR₃—(CH₂)ₙ—NR₄R₅; —O—(CH₂)ₙ—NR₄R₅, —NR₃—(CH₂)ₙ—N⁺R₆R₇R⁸ and —O—(CH₂)ₙ—N⁺R₆R₇R₈,
R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
R₃ is selected from the group consisting of H; (C₁-C₆)-alkyl; (C₁-C₆)-alkyl-aryl;
R₄ and R₅, identical or different, are selected from the group consisting of H; (C₁-C₆)-alkyl; aryl and (C₁-C₆)-alkyl-aryl;
R₆, R₇ and R₈, identical or different, are selected from the group consisting of (C₁-C₆)-alkyl; aryl and (C₁-C₆)-alkyl-aryl;
—Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
R₉ and R₁₀, identical or different, are selected from the group consisting of H, (C₁-C₆)-alkyl, aryl and (C₁-C₆)-alkyl-aryl;
R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl n=0, 2, 3, 4, 5 or 6, with the proviso that at least one of W, X and Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈, for use in the treatment of Multiple Myeloma (MM).

The invention also relates to a pharmaceutical composition comprising, in a pharmaceutical acceptable vehicle, the compounds of the invention of formula (I), enantiomers, mixture of enantiomers, diastereoisomers and mixture of diastereoisomers thereof, for use in a method for treating subjects having Multiple Myeloma (MM).

In a particular embodiment, the pharmaceutical composition of the present invention is for use in a method for treating subjects likely to display an MM relapse and/or death, or subjects refractory or resistant to a first line treatment. Said subjects are also named subjects with poor outcome.

By ‘subjects likely to display and MM relapse and/or death’, it means ‘subjects having a poor outcome’.

By ‘subjects refractory or resistant to a first line treatment’, it means subjects refractory or resistant to a first line treatment that is distinct from salinomycin treatment according to the present invention.

By ‘first line treatment’ or ‘first line-therapy’, it means the first treatment given for a disease. It is often part of a standard set of treatments, such as surgery followed by chemotherapy and radiation. When used by itself, first-line therapy is the one accepted as the best treatment. If it doesn't cure the disease or it causes severe side effects, other treatment may be added or used instead.

Another subject-matter of the invention concerns a pharmaceutical product comprising:
(i) a compound of formula (I) according to the invention and
(ii) another anti-cancer agent or cellular therapy for treating MM selected in the group consisting of agents used either in chemotherapy, agents used in targeted treatment, agents used in immune therapies or combinations thereof, in particular selected in the group consisting of proteasome inhibitors (PIs), immunomodulatory agents in particular immunomodulatory drugs (IMiDs), DNA methyltransferase inhibitor, chemo drugs, inhibitors of nuclear export in particular exportin 1 inhibitor, corticosteroids, histone deacetylase (HDAC) inhibitors, therapeutic monoclonal antibodies (moAb) in particular anti-CD38, anti-SLAMF7, and/or anti BCMA, antibodies drug conjugates (ADC), bispecific T-cell engager (BiTES), MCL1 inhibitors or other BH3 mimetics, CART-T cells and combinations thereof,
as combination product for simultaneous, separate or staggered use in the treatment of MM.

The present invention also relates to a pharmaceutical product comprising:
(i) a compound of formula (I) of the invention wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl,
(ii) an anticancer agent selected in the group consisting of lenalidomide, pomalidomide (immunomodulatory agents), melphalan (chemo-drug), carfilzomib (proteasome inhibitor), AZD-5991 (MCL1 inhibitor), and combinations thereof and
(iii) optionally another anticancer agent or cellular therapy for treating MM selected in the group consisting of proteasome inhibitors (Pis), immunomodulatory agents, chemo drugs, inhibitors of nuclear export in particular exportin 1 inhibitor, corticosteroids, histone deacetylase (HDAC) inhibitors, therapeutic monoclonal antibodies (moAb) in particular anti-CD38, anti-SLAMF7, and/or anti BCMA, antibodies drug conjugates (ADC), bispecific T-cell engager (BiTES), MCL1 inhibitors or other BH3 mimetics, CART-T cells and combinations thereof.

Another subject matter of the invention is a pharmaceutical composition according to the invention or a pharmaceutical product according to the invention, for use in the treatment of a MM subject that has been identified as having a poor outcome by a method comprising the steps of:
a) Measuring the expression level of at least 3, in particular at least 5 genes and/or proteins encoded by the said at least 3, in particular the said at least 5 genes selected in the group consisting of CYBRD1, EPAS1, FBXL5, PPOX, SLC39A14, and STEAP1 involved in the iron metabolism, in a biological sample obtained from said subject;
b) Calculating a score value from said expression level obtained at step a)
c) Classifying and identifying the said subject as having a poor outcome according to the score value in comparison to a predetermined reference value (PRV).

The present invention also concerns an in vitro method for identifying MM subject with a poor outcome that may benefit from a therapeutic treatment comprising a compound of formula (I), enantiomers, mixture of enantiomers, diastereoisomers and mixture of diastereoisomers thereof as defined in the present invention or a pharmaceutical composition as defined in the invention or a pharmaceutical product as defined in the invention, comprising the steps of:
a) Measuring the expression level of at least 3, in particular at least 5 genes and/or proteins encoded by the said at least 3, in particular the said at least 5 genes selected in the group consisting of CYBRD1, EPAS1, FBXL5, PPOX, SLC39A14, and STEAP1 involved in the iron metabolism, in a biological sample obtained from said subject;
b) Calculating a score value from said expression level obtained at step a)
c) Classifying and identifying the said subject as having a poor outcome according to the score value in comparison to a predetermined reference value (PRV).

### Definitions

Salinomycin is a monocarboxylic polyether possessing ionophoric properties of the following formula:

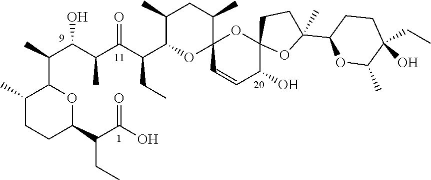 [SMILES: CCC(C(=O)O)[C@H]1CC[C@H](C)C([C@@H](C)[C@H](O)[C@H](C)C(=O)[C@H](CC)[C@H]2O[C@]3(C=C[C@@H](O)[C@]4(CC[C@@](C)([C@H]5CC[C@](O)(CC)[C@H](C)O5)O4)O3)[C@H](C)C[C@@H]2C)O1]

and the compounds used according to the present invention in Acute Myeloid Leukemia (AML) treatment are 9- and/or 11- and/or 20-amino derivatives of salinomycine, in particular 20-amino derivatives of salinomycine as disclosed in the patent application WO2016/038223. These derivatives of salinomycin are synthetic small molecules chemically derived from salinomycin exhibiting a more potent activity and potentially lower toxicity against healthy cells.

By ‘derivatives thereof’ according to the invention, it means synthetic small molecules chemically derived from salinomycin exhibiting a more potent activity and potentially lower toxicity against healthy cells.

The expression “multiple myeloma” (MM) refers to the multiple myeloma disease such as defined by class C90.0 in accordance with the International Classification of Diseases World Health Organisation Classification (10th revised edition; 2016).

The term ‘subject’ or ‘patient’ or ‘individual’ refers to a human subject, whatever its age or sex. The subject is affected by a Multiple Myelome (MM). The subject may be already subjected to a treatment, by any chemotherapeutic agent, or may be untreated yet.

The term ‘MM subject’ refers to a subject having MM originating from a population of MM subjects, from early to late stage of MM, the said subjects undergoing or not undergoing a therapeutic treatment.

In a particular embodiment, the MM subject is likely to display an MM relapse and/or death, or is refractory or resistant to a first line treatment, such MM subject is also named as having a ‘poor outcome’ or ‘poor prognosis’. In another embodiment, the MM subject is likely to display an MM relapse.

The term “outcome” refers to the survival, the relapse or the death of the subject. The outcome may relate to disease-free survival (DFS), event free survival (EFS) or overall survival (OS), as defined within the state of the art. Illustratively, a “bad or poor outcome” may refer to a disease relapse or death of the subject. Oppositely, a “good outcome” may refer to survival of the subject, with or without relapse episode.

Within the scope of the present invention, a “biological sample” refers to a biological sample obtained, reached, collected or isolated from a subject, in vivo or in situ. Such samples may be, but not limited to, organs, tissues, fractions and cells isolated from a subject. For example, suitable biological samples include but are not limited to a cell culture, a cell line, a tissue biopsy such as a bone marrow aspirate, a biological fluid such as a blood, pleural effusion or a serum sample, and the like.

In certain embodiments, the preferred biological sample includes but is not limited to a blood sample, a tissue biopsy, including a bone marrow aspirate.

In some embodiments, the biological sample may be a crude sample.

In some other embodiments, the biological sample may be purified to various degrees prior to storage, processing, or measurement.

By ‘first-line treatment’ or ‘first-line therapy’, it means treatment regimen or regimens that are generally accepted by the medical establishment for initial treatment of a given type and stage of cancer. Second-line therapies are those tried when the first ones do not work adequately, ie have some limited efficacy, or produce unacceptable side effects, damage organs in the body.

The term “treating” or “treatment” means stabilizing, alleviating, curing, or reducing the progression of the MM.

The ‘iron-score’ according to the invention is a GEP (Gene Expression Profile)-based iron-score; it is defined by the sum of the beta coefficients of the Cox model for each prognostic gene, weighted by ±1 according to the patient signal above or below the probe set Maxstat value.

By ‘prognosis marker’, it means a marker relevant to assess the outcome of the subject. In particular the expression profile or expression level of the genes and/or proteins identified in the present invention as being differentially expressed in MM subjects, represents a prognosis marker that permits to identify subjects having ‘good prognosis’ from subjects having ‘bad prognosis’.

The 6 genes for MM identified to be informative to assess the outcome of the subject are also named in the disclosure as ‘genes of interest’ or ‘prognosis genes’ or ‘prognostic genes’.

By ‘good prognosis’ or ‘good outcome’ according to the present invention, it means the survival of the subject.

By ‘poor prognosis’ or ‘poor outcome’ according to the present invention, it means the ‘disease relapse’ or the ‘death’ of the subject.

By a “reference sample”, it is meant a biological sample of a patient whose clinical outcome is known (i.e., the duration of the disease-free survival (DFS), or the event free survival (EFS) or the overall survival (OS) or both). Preferably, a pool of reference samples comprises at least one (preferably several, more preferably at least 5, more preferably at least 6, at least 7, at least 8, at least 9, at least 10) ‘good outcome’ patient(s) and at least one (preferably several, more preferably at least 6, at least 7, at least 8, at least 9, at least 10) ‘bad outcome’ patient(s). The highest the number of reference samples, the better for the reliability of the method of prediction of the outcome of the subject tested according to the invention. Said reference samples (collection samples of MM subjects) for which expression profile of the prognosis genes is evaluated, permits to measure predetermined reference values (PREV and PREL as further disclosed), which are used for comparison purposes.

### BRIEF DESCRIPTION OF THE FIGURES

FIG. 1: Depmap representation of STEAP1 gene in MM

FIG. 2: The iron gene risk score predicts overall and event free survival in patients with MM.

(A) Kaplan-Meier estimates of the overall and event free survival in newly-diagnosed patients from the TT2 cohort (N=345) with high and low iron score values. The prognostic value of the iron score was confirmed in two other independent cohorts of newly diagnosed patients (B) HM cohort (N=206) and (C) TT3 cohort (N=158), as well as (D) in a cohort of 188 patients at relapse treated with bortezomib monotherapy (Mulligan cohort N=188).

FIG. 3: Ironomycin and AM-23 kills HMCL cells with nanomolar concentration

The panel of 17 HMCL cell lines were incubated with increasing concentrations of Ironomycin (A), AM23 (B) or vehicle for 96H.

FIG. 4: Ironomycin induces HMCL cell death

XG-1, XG-7 and OPM-2 cell lines were treated with IC₅₀ of ironomycin during 72H. Apoptosis induction was monitored by Annexin V staining (XG-1 and XG-7) or PARP-cleavage and analyzed by flow cytometry (A).

XG7 and OPM-2 cells were pretreated at least 30 minutes by 20 μM of Q-VD-Oph as pan caspase inhibitor before treatment. Cells were treated as indicated during 48H, effect of Ironomycin on cell death induction was analyzed using AnnexinV-PE staining by flow cytometry (B). N=4.

OPM-2 cells were treated as indicated during 48H and pan-caspase, caspase3/7 and caspase 9 activity were measured by luminometry. 4-OH-cyclophosphamide (80 μM) was used as a positive control (C).

Results represent the mean of percentage±SD of three independent experiments.

Statistical significance was tested using t-test of pairs: * P<0.05, ** P<0.01 *** P<0.001, **** P<0.0001 and NS: non-significant.

FIG. 5: Ironomycin affects MM cell division.

XG-1, XG-7 and OPM-2 cells were incubated with vehicle or with IC₅₀ Ironomycin for 72 hours. Cell cycle was analyzed using flow cytometry, S phase was stained by an anti-BrdU antibody after BrdU incorporation and DNA content was strained by 4′,6-diamidino-2-phénylindole (DAPI). Histograms represent the mean percentage and SD of each cell cycle phase of three independent experiments. * and ** indicate a significant difference of P<0.05 and P<0.01, respectively with paired student t-test.

FIG. 6: Apoptosis induced by Ironomycin was not reversed by iron supplementation

XG-7 and OPM-2 cell line was pre-incubated with or without 80 μM of deferasirox or 100 nM of Ironomycin, for 4 hours followed by 72H incubation in presence or absence of FeCl3 (100 μM). Apoptosis was assessed using Annexin V-PE staining by flow cytometry.

Iron supplementation significantly inhibited the effect of iron chelators on MCL cells apoptosis (P<0.01 and P<0.001 for Deferasirox treatment). However, iron supplementation did not affect ironomycin-induced MCL cell cytotoxicity.

FIG. 7: Ironomycin induces DNA damage response

Cells were treated with iron chelators (80 μM Deferasirox), and Ironomycin (IC₅₀ and 200 nM) during 24H. Protein levels of Phospho-H2A.X (S139) were analyzed by western blot and normalized by the histone 3 protein level.

FIG. 8: Ironomycin deregulates MMSET, MYC and histone methylation

Cells were treated with iron chelators (80 μM Deferasirox), and Ironomycin (IC₅₀ and 200 nM) during 24H. Protein levels of MMSET (isoforms I and II), MYC, H3K36me3 and H3K27me3 were analyzed by western blot. MMSET (isoforms I and II) and MYC signal were normalized by α-tubulin protein level, H3K36me3 and H3K27me3 signal were normalized by total histone 3 protein level.

FIG. 9: Ironomycin has low toxicity on hematopoietic progenitors compared to AM23

Hematopoietic progenitor colony-forming units assay were performed with CD34+ cells from apheresis of 5 donors. Cells were cultured in hydroxyl-methyl-cellulose medium with or without conventional chemotherapy or Ironomycin (A) or AM-23 (B). CFU-C, CFU-E/BFU-E and CFU-GM, -G, -M were counted after 14 days culture. N=5.

FIG. 10: Assays on primary MM cells of patients

Primary MM cells were treated with Ironomycin (A) or AM-23 (B) and incubated during 96H with IL-6. The toxicity on MM cells and non-MM cells was analyzed by flow cytometry and expressed in % of control.

Each individual experiments of primary MM cells are presented on the FIG. 9C.

N=5, Median+/−IQR, t-test of pairs. * P<0.05, ** P<0.01, *** P<0.001, **** P<0.0001.

FIG. 11A: Synergistic effect of Ironomycin combined with melphalan.

XG-7 or XG2 WT (XG2 neg) or XG2 melphalan resistant (XG2 Melph R) cell lines were respectively treated with increasing concentrations of ironomycin combined with melphalan during 96 h and cell viability was tested by ATP quantification to obtain the viability matrix. The synergy matrix was calculated as described in Material and Methods.

FIG. 11B: Synergistic effect of Ironomycin combined with lenalidomide.

XG-7cells were treated with increasing concentrations of ironomycin combined with lenalidomide during 96 h and cell viability was tested by ATP quantification to obtain the viability matrix. The synergy matrix was calculated as described in Material and Methods.

FIG. 11C: Synergistic effect of Ironomycin with pomalidomide.

XG-7cells were treated with increasing concentrations of ironomycin combined with pomalidomide during 96 h and cell viability was tested by ATP quantification to obtain the viability matrix. The synergy matrix was calculated as described in Material and Methods.

FIG. 11D: Additive effect of Ironomycin combined with carfilzomib.

XG-7cells were treated with increasing concentrations of ironomycin combined with carfilzomib during 96 h and cell viability was tested by ATP quantification to obtain the viability matrix. The synergy matrix was calculated as described in Material and Methods.

FIG. 11E: Additive effect of Ironomycin combined with AZD-5991 (MCL-1 inhibitor).

XG-7cells were treated with increasing concentrations of ironomycin combined with AZD-5991 during 96 h and cell viability was tested by ATP quantification to obtain the viability matrix. The synergy matrix was calculated as described in Material and Methods

FIG. 12: Primary bone marrow samples of 7 MM patients were incubated with 5 mmol/L Rho-Nox1 Fe2⁺ probe. The MFI was assessed by flow cytometry in normal B cells, normal plasma cells and malignant plasma cells.

### DETAILED DESCRIPTION OF EMBODIMENTS OF THE INVENTION

Multiple myeloma (MM) is the second most common blood cancer, and is affecting the plasma cells. In subjects having multiple myeloma, malignant clonal plasma cells accumulate within the bone marrow. These aberrant cells are ‘diluting’ the normal plasma cells that are devoted to fight infections. The malignant plasma cells also produce abnormal proteins, such as M protein, which may cause solid tumors, damage the kidneys, and impair immune system function. In some cases, the malignant cells may cause a single tumor, called a solitary plasmacytoma, but if multiple tumors are formed, and then the disease is called multiple myeloma (MM).

The inventors have demonstrated in the examples later in the description that compounds of formula (I) according to the invention, in particular Ironomycin (AM5) and AM-23, are able to kill MM cells with nanomolar concentration and may be combined with anticancer compound for MM with a synergistic effect.

So the present invention concerns a compound of formula (I), enantiomers, mixture of enantiomers, diastereoisomers and mixture of diastereoisomers thereof:

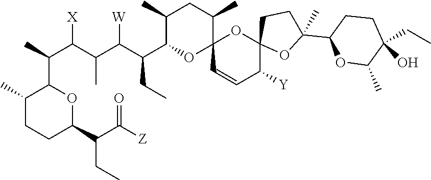

wherein:
—W is selected from the group consisting of ═O; —NR₁R₂; —NR₃—(CH₂)ₙ—NR₄R₅; —O—(CH₂)ₙ—NR₄R₅; —NR₃—(CH₂)ₙ—N⁺R₆R₇R⁸ and —O—(CH₂)ₙ—N⁺R₆R₇R₈;
—X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ—N⁺R₆R₇R⁸ and —O—(CH₂)ₙ—N⁺R₆R₇R₈,
—Y is selected from the group consisting of —OH; —N—OH; —NR₁R₂; —NR₃—(CH₂)ₙ—NR₄R₅; —O—(CH₂)ₙ—NR₄R₅; —NR₃—(CH₂)ₙ—N⁺R₆R₇R⁸ and —O—(CH₂)ₙ—N⁺R₆R₇R₈,
R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
R₃ is selected from the group consisting of H; (C₁-C₆)-alkyl; (C₁-C₆)-alkyl-aryl;
R₄ and R₅, identical or different, are selected from the group consisting of H; (C₁-C₆)-alkyl; aryl and (C₁-C₆)-alkyl-aryl;
R₆, R₇ and R₈, identical or different, are selected from the group consisting of (C₁-C₆)-alkyl; aryl and (C₁-C₆)-alkyl-aryl; —Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
R₉ and R₁₀, identical or different, are selected from the group consisting of H, (C₁-C₆)-alkyl, aryl and (C₁-C₆)-alkyl-aryl;
R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl n=0, 2, 3, 4, 5 or 6,
with the proviso that at least one of W, X and Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
for use in the treatment of Multiple Myeloma (MM).

In the sense of the present invention:
“(C₁-C₁₆)-alkyl” designates an optionally substituted acyclic, saturated, linear or branched hydrocarbon chain comprising 1 to 16 carbon atoms. Examples of (C₁-C₁₆)-alkyl groups include methyl, ethyl, propyl, butyl, pentyl, hexyl, heptyl, octyl, and dodecyl;
“—(C₃-C₁₆)-alkenyl” designates an optionally substituted acyclic, saturated, linear or branched hydrocarbon chain comprising 3 to 16 carbon atoms, at least two of which are linked via a double bond. Examples of “—(C₃-C₁₆)-alkenyl” include propenyl, butenyl, pentenyl or hexenyl;
“C₃-C₁₆)-alkynyl” designates an optionally substituted acyclic, saturated, linear or branched hydrocarbon chain comprising 3 to 16 carbon atoms, at least two of which are linked via a triple bond. Examples of “—(C₃-C₁₆)-alkynyl” include propynyl, butynyl, pentynyl or hexynyl;
“(C₃-C₁₆)-cycloalkyl” designates an optionally substituted cyclic, saturated hydrocarbon chain comprising 1 to 16 carbon atoms. Examples of (C₃-C₁₆)-cycloalkyl groups include cyclopropyl, cyclobutyl, cyclopentyl, cyclohexyl, cycloheptyl, cyclooctyl, and cyclododecyl. Advantageously, (C₃-C₁₆)-cycloalkyl group is selected from optionally substituted cyclopropyl, cyclobutyl and cyclopentyl;
“aryl” designates an aromatic, monocyclic ring that may be fused with a second saturated, unsaturated or aromatic ring. The term aryl includes, without restriction to the following examples, phenyl, indanyl, indenyl, naphtyl, anthracenyl, phenanthrenyl, tetrahydronaphtyl, and dihydronaphtyl. The preferred aryl are those comprising one six-membered aromatic ring. The aryl group may be substituted with one or more groups independently selected from the group consisting of alkyl, alkoxy, halogen, hydroxyl, amino, nitro, cyano, trifluoro, carboxylic acid or carboxylic ester. Examples of substituted phenyl groups are methoxyphenyl, dimethoxyphenyl, trimethoxyphenyl, fluorophenyl and trifluoromethylphenyl;
“—(C₁-C₆)-alkyl-aryl” designates in the sense of the present invention an aryl group, as defined above, linked to the rest of the molecule by an alkyl chain containing 1 to 6 carbon atoms. Advantageously, the ““-(C₁-C₆)-alkyl-aryl is a substituted or unsubstituted benzyl. Examples of substituted benzyl groups include hydroxybenzyl, methoxybenzyl, cyanobenzyl, nitrobenzyl or fluorobenzyl;
“heteroaryl” designates a mono- or polycyclic aryl as defined above where one or more carbon atoms have been replaced with one or more heteroatoms selected from the group consisting of N, O and S. Examples of heteroaryl groups include furyl, thienyl, imidazolyl, pyridyl, pyrrolyl, N-alkyl pyrrolyl, pyrimidinyl, pyrazinyl, tetrazolyl, triazolyl and triazinyl. The heteroaryl group may be substituted with one or more groups independently selected from the group consisting of alkyl, alkoxy, halogen, hydroxyl, amino, nitro, cyano, trifluoro, carboxylic acid or carboxylic ester. Preferred heteroaryls are those having 5 or 6 atoms in the ring, such as indolyl, pyrrolyl, pyridinyl, pyrrazolyl, triazolyl, furanyl or thienyl.
“—(C₁-C₆)-alkyl-heteroaryl” designates in the sense of the present invention an heteroaryl group, as defined above, linked to the rest of the molecule by an alkyl chain containing 1 to 6 carbon atoms. Advantageously, the “—(C₁-C₆)-alkyl-heteroaryl” is a substituted or (C₁)-alkyl-heteroaryl.

The term “optionally substituted” as used herein means that any of the hydrogen atoms can be replaced by a substituent selected from the group consisting of alkyl, alkoxy, halogen, hydroxyl, amino, nitro, cyano, trifluoro, carboxylic acid or carboxylic ester Advantageously, R₁ and R₂, identical or different, are selected from the group consisting of H; (C₁-C₁₆)-alkyl, advantageously (C₃-C₁₄)-alkyl, more advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

Advantageously, R₁ and R₂ are not both H.

More advantageously, R₁ is H and R₂ is selected from the group consisting of (C₁-C₁₆)-alkyl, advantageously (C₃-C₁₄)-alkyl, more advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

Advantageously, R₃ is selected from the group consisting of H and (C₁-C₆)-alkyl. Preferably, R₃ is H.

Advantageously, R₄ and R₅, identical or different, are selected from the group consisting of H and (C₁-C₁₆)-alkyl. More advantageously, R₄ and R₅ are H or (C₁-C₆)-alkyl. Preferably, R₄ and R₅ are identical. In one advantageous embodiment, the group —(CH₂)ₙ—NR₄R₅ is selected from the group consisting of —(CH₂)₂—N(CH₃)₂, —(CH₂)₃—N(CH₃)₂, —(CH₂)₂—NH₂ and —(CH₂)₃—NH₂.

Advantageously, R₆, R₇ and R₈, identical or different, are selected from the group consisting of (C₁-C₆)-alkyl; and aryl. More advantageously, R₆, R₇ and R₈ are (C₁-C₆)-alkyl. Preferably, R₆, R₇ and R₈ are identical. In one advantageous embodiment, the group —(CH₂)ₙ—N⁺R₆R₇R₈ is selected from the group consisting of —(CH₂)₂—N⁺(CH₃)₃, and —(CH₂)₃—N⁺(CH₃)₃.

Advantageously, Z is OH, OOH, NHNH₂, NHOH, or NH₂OH, preferably OH.

In an advantageous embodiment according to the present invention, the compound of formula (I) is a monoamine derivative of salinomycine, and only one of W, X or Y is a —NR₁R₂; —NR₃—(CH₂) n-NR₄R₅; —O—(CH₂)ₙ—NR₄R₅; —NR₃—(CH₂)ₙ—N⁺R₆R₇R₈; or —O—(CH₂)ₙ—N⁺R₆R₇R₈ group, and
W, X, Y, Z, R₁, R₂, R₃, R₄, R₅, R₆, R₇, R₈ and n are as defined in formula (I).

The compound of formula (I) used in the present invention is advantageously a 20-amino derivative of salinomycine of formula (Ic3) as disclosed in WO2016/038223:

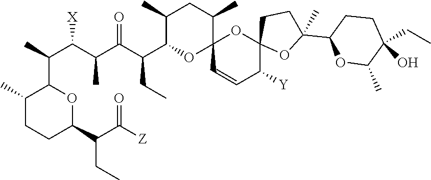

wherein:
X is selected from the group consisting of OH and ═O,
Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈; and —O—(CH₂)ₙ—N⁺R₆R₇R₈; and
Z, R₁, R₂, R₃, R₄, R₅, Re, R₇, R₈ and n are as defined in formula (I).

In one advantageous embodiment, in the compounds of formula (Ic3), X is OH, Z is OH, and Y is —NR₁R₂. More advantageously, R₁ is H and R₂ is selected from the group consisting of (C₁-C₁₆)-alkyl, advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

In another embodiment, X is ═O, Y is selected from the group consisting of ═N—OH and NR₁R₂ and Z is NHOH. Advantageously, X is ═O, Y is NR₁R₂ and Z is NHOH. More advantageously, R₁ is H and R₂ is CH₂-pyridinyl, preferably CH₂-(2-pyridinyl). Alternatively, R₁ is H and R₂ is (C₃-C₁₆)-cycloalkyl or (C₃-C₁₆)-alkynyl.

In a particular embodiment, when Z is —NHOH, W is ═O and X is —OH, then Y is not a propargyl group.

In a particular embodiment, when Z is —OH, W is ═O and X is —OH, then Y is not NCH₂CH₂N(CH₃)₂.

The compounds of formula (I) used according to the invention, and methods of synthesis of said compounds are disclosed in the patent application WO2016/038223.

In a particular and preferred embodiment, the compound of formula (I) is as defined above, wherein X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, (C₃-C₁₆)-cycloalkyl, (C₁-C₆)-alkyl-aryl and (C₁-C₆)-alkyl-heteroaryl.

In a more preferred embodiment, the compound of formula (I) is as defined above, wherein X is OH, Z is OH and Y is NR₁R₂ where R₁ is H and R₂ is selected from the group consisting of (C₈-C₁₄)-alkyl; (C₃-C₅)-alkenyl; (C₃-C₅)-alkynyl, (C₃-C₆)-cycloalkyl, benzyl, and CH₂-pyridynyl, preferably (C₃-C₅)-alkynyl.

In a particular embodiment, W is ═O, X is —OH, Y is —NR₁R₂ preferably with R₁ being H and R₂ being (C₃-C₁₆)— alkynyl, preferably propargyl, and Z is —OH. Such compound is also named Ironomycin or compound AM5 as disclosed in the patent application WO2016/038223.

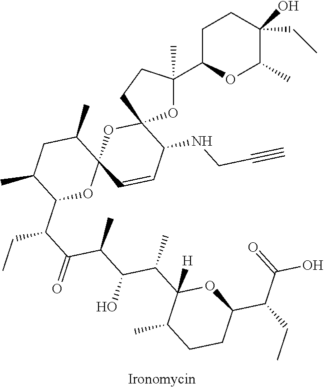 [SMILES: C#CCN[C@@H]1C=C[C@]2(O[C@H]([C@@H](CC)C(=O)[C@@H](C)[C@@H](O)[C@H](C)[C@@H]3O[C@@H]([C@@H](CC)C(=O)O)CC[C@@H]3C)[C@@H](C)C[C@H]2C)O[C@@]12CC[C@@](C)([C@H]1CC[C@](O)(CC)[C@H](C)O1)O2]

In a particular embodiment, W is ═O, X is —OH, Y is —NR₁R₂ preferably with R₁ being H and R₂ being (C₃-C₁₆)-cycloalkyl, preferably cyclopropyl, and Z is —OH. Such compound is also named AM23 as disclosed in the patent application WO2016/038223.

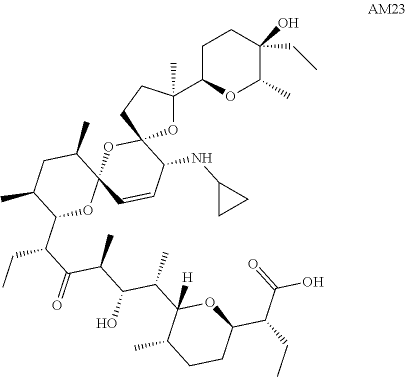 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NC3CC3)[C@]3(CC[C@@](C)([C@H]4CC[C@](O)(CC)[C@H](C)O4)O3)O2)[C@H](C)C[C@@H]1C]

In another particular embodiment, W is ═O, X is OH, Z is OH, and Y is NR₁R₂ where RH is H and R₂ is a (C₃-C₆)-cycloalkyl group, in particular a substituted cyclopropyl as disclosed hereunder:

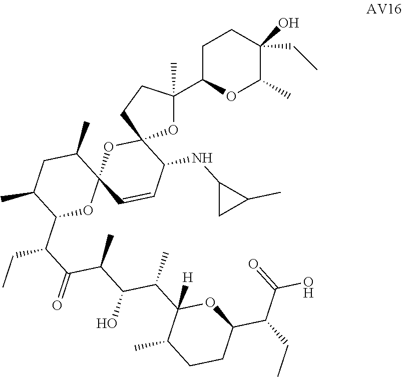 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NC3CC3C)[C@]3(CC[C@@](C)([C@H]4CC[C@](O)(CC)[C@H](C)O4)O3)O2)[C@H](C)C[C@@H]1C]

In another particular embodiment, W is ═O, X is OH, Z is OH, and Y is NR₁R₂ where R₁ is H and R₂ is a (C₁-C₆)-alkyl-aryl group, in particular a benzyl group substituted by an hydroxy, as disclosed hereunder:

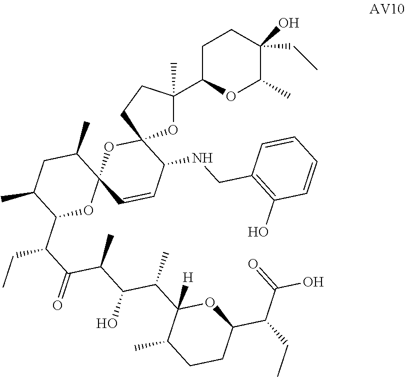 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NCc3ccccc3O)[C@]3(CC[C@@](C)([C@H]4CC[C@](O)(CC)[C@H](C)O4)O3)O2)[C@H](C)C[C@@H]1C]

In another particular embodiment, W is ═O, X is OH, Z is OH, and Y is NR₁R₂ where R₁ is H and R₂ is a (C₁-C₆)-alkyl-pyridyl group, in particular a CH₂-pyridinyl group, as disclosed hereunder:

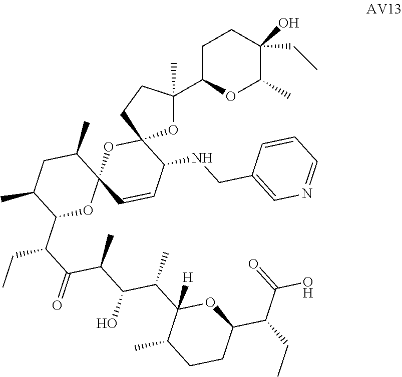 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NCc3cccnc3)[C@]3(CC[C@@](C)([C@H]4CC[C@](O)(CC)[C@H](C)O4)O3)O2)[C@H](C)C[C@@H]1C]

The compounds AM5, AM23, AV10, AV13 and AV16, preferably AM5 are particular and preferred compounds used in the pharmaceutical composition, pharmaceutical product and therapeutic uses disclosed hereunder.

### Pharmaceutical Composition for Use in the Treatment of MM Subjects

Another object of the invention is a pharmaceutical composition comprising in a pharmaceutical acceptable vehicle, at least a compound of formula (I) as disclosed above, for use in a method for treating subjects having Multiple Myeloma (MM).

In a particular embodiment, the compound of formula (I) is with W is ═O, X is OH, Z is OH, and Y is NR₁R₂ where R₁ is H and R₂ is selected from the group consisting of (C₁-C₁₆)-alkyl, (C₃-C₁₆)-alkenyl, (C₃-C₁₆)-alkynyl, (C₃-C₁₆)-cycloalkyl, (C₁-C₆)-alkyl-aryl and (C₁-C₆)-alkyl-heteroaryl.

In a preferred embodiment, the compound of formula (I) is with W is ═O, X is OH, Z is OH, and Y is NR₁R₂ where R₁ is H and R₂ is selected from the group consisting of (C₈—C₁₄)-alkyl; (C₃-C₅)-alkenyl; (C₃-C₅)-alkynyl, (C₃-C₆)-cycloalkyl, benzyl, and CH₂-pyridynyl, preferably (C₃-C₅)-alkynyl.

In a more preferred embodiment, the compound of formula (I) is with W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl, and most preferably propargyl.

The pharmaceutical composition for use according to the invention comprises at least one compound of formula (I) as defined above, a pharmaceutical salt, solvate or hydrate thereof, and at least one pharmaceutically acceptable excipient.

For the purpose of the invention, the term ‘pharmaceutically acceptable’ is intended to mean what is useful to the preparation of a pharmaceutical composition, and what is generally safe and non-toxic, for a pharmaceutical use.

The term pharmaceutically acceptable salt, hydrate of solvate is intended to mean, in the present invention, a salt of a compound which is pharmaceutically acceptable, as defined above, and which possesses the pharmacological activity of the corresponding compound.

Such salts comprise:
hydrates and solvates,
acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

The pharmaceutical compositions for use according to the invention can be intended to oral, sublingual, subcutaneous, intramuscular, intravenous, transdermal, topical or rectal administration. The active ingredient can be administered in unit forms for administration, mixed with conventional pharmaceutical carriers, to animals or to humans. When a solid composition is prepared in the form of tablets, the main active ingredient is mixed with a pharmaceutical vehicle and other conventional excipients known to those skilled in the art.

The compounds of the invention can be used in a pharmaceutical composition at a dose adapted by the person skilled in the art to be administered in only one dose once a day or in several doses along the day, for example twice a day.

In a particular embodiment, the said pharmaceutical composition is used in a method for treating subjects likely to display an MM relapse and/or death, or subjects refractory or resistant to a first line treatment.

### Pharmaceutical Product (Also Named “Combination Product”) and Uses in Treatment of MM Subjects

An aspect of the invention also relates to a compound of formula (I) as disclosed herein, optionally combined with an anticancer treatment, in particular a conventional anti-MM treatment, for use in treating a multiple myeloma (MM) subject in need thereof.

In a particular embodiment, the present invention relates to a pharmaceutical product comprising:
(i) a compound of formula (I) according to the invention and
(ii) another anti-cancer agent selected from the group consisting of agents used either in chemotherapy, in targeted treatments, in immune therapies, and combinations thereof, as combination product for simultaneous, separate or staggered use in the treatment of MM, in particular in MM subjects having a poor outcome.

The expression “pharmaceutical product” or “combination product” according to the invention means herein that the compound of formula (I) used in the present invention is administered to the subject treated before, during (including concurrently with-preferably co-formulated with) and/or after treatment of the subject with the other anti-cancer drug. The formulations may conveniently be presented in unit dosage form by methods known to those skilled in the art. Preferably, the kit-of-parts contains instructions indicating the use of the dosage form to achieve a desirable affect and the amount of dosage form to be taken over a specified time period. The compound of formula (I) may be combined with one, two or more other anti-cancer agents.

It shall be understood that a combined treatment with two or more active ingredients, among those described herein, does not mean in all cases that the said two or more active ingredients are administered simultaneously to an individual in need thereof.

In some embodiments, the two or more active ingredients of the said combined treatment are combined in a single pharmaceutical composition before administering the said pharmaceutical composition to the said individual in need thereof. These embodiments include those wherein the said combined treatment involves three or more active ingredients and wherein two or more active ingredients are combined in a single pharmaceutical composition.

In some other embodiments, each of the active ingredients is comprised in a separate pharmaceutical composition and the said individual is administered with each of the said separate pharmaceutical compositions.

When two or more separate pharmaceutical compositions are to be administered to the said subject in need thereof, then the said pharmaceutical compositions may be administered successively in a short period of time, e.g., a one hour or less period of time.

In some other embodiments, the said separate pharmaceutical compositions may be administered in a longer period of time, at time intervals of more than one hour.

In some further embodiments, the time intervals at which the said separate pharmaceutical compositions are administered to the said subject in need thereof may significantly vary. Illustratively, a first pharmaceutical composition is administered twice a day, whereas a second pharmaceutical composition is administered daily. Still illustratively, a first pharmaceutical composition is administered daily and a second pharmaceutical composition is administered weekly.

By ‘agents used in chemotherapy’, it means drugs also named ‘chemo drugs’ able to stop the growth of cancer cells, either by killing the cells or by stopping them from dividing.

By ‘agents used in targeted treatments’ according to the invention, it means drugs or other substances able to identify and attack specific types of cancer cells with less harm to normal cells. Some targeted therapies block the action of certain enzymes, proteins, or other molecules involved in the growth and spread of cancer cells. Other types of targeted therapies help the immune system kill cancer cells or deliver toxic substances directly to cancer cells and kill them. Targeted therapy may have fewer side effects than other types of cancer treatment. Most targeted therapies are either small molecule drugs or monoclonal antibodies.

By ‘agents used in immune therapies’ according to the invention, it encompasses ‘immunomodulatory agents’ able to stimulate or suppress the immune system to help the body fight cancer. Some types of immunotherapy only target certain cells of the immune system. Others affect the immune system in a general way. Types of immunotherapy include as examples cytokines, and some monoclonal antibodies.

In a particular embodiment, the present invention also relates to a pharmaceutical product or combination product comprising:
(i) a compound of formula (I) as defined above and
(ii) another anti-cancer agent or cellular therapy for treating MM selected in the group consisting of proteasome inhibitors (Pis), immunomodulatory agents in particular immunomodulatory drugs (IMiDs), DNA methyltransferase inhibitor, chemo drugs, inhibitors of nuclear export in particular exportin 1 inhibitor, corticosteroids, histone deacetylase (HDAC) inhibitors, therapeutic monoclonal antibodies (moAb) in particular anti-CD38, anti-SLAMF7, and/or anti BCMA, antibodies drug conjugates (ADC), bispecific T-cell engager (BiTES), MCL1 inhibitors or other BH3 mimetics, CART-T cells and combinations thereof as combination product for simultaneous, separate or staggered use in the treatment of MM.

The review of Yamamoto et al. (2021) discloses the current immune therapies and new therapies to treat MM.

Non limitative examples of such compounds are illustrated hereunder:

### Proteasome Inhibitors

In some embodiments, anticancer compounds may include a proteasome inhibitor, in particular selected in a group comprising bortezomib, carfilzomib and ixazomib.

### Immunomodulatory Agents

In some embodiments, the immunomodulatory agent is an immunomodulatory drug, also referred as “IMiD”, i.e. a structural and functional analogue of thalidomide. Illustratively, IMiD compounds encompass lenalidomide, pomalidomide, and derivative thereof, as well as compounds disclosed by Knight (2005). In certain embodiments, the immunomodulatory agent is selected in a group comprising thalidomide, lenalidomide, pomalidomide and derivatives thereof. Within the scope of the invention, the term “derivative of is intended to refer to a compound having structural and functional analogy with a compound of interest.

In some embodiments, the immunomodulatory agent is selected in a group comprising thalidomide, lenalidomide, pomalidomide and a derivative thereof, in particular lenalidomide.

### DNA Methyltransferase Inhibitor

In some other embodiments, the DNA methyltransferase inhibitor is selected in a group consisting of 5-azacytidine, zebularine, caffeic acid, CC-486 (azacytidine), chlorogenic acid, epigallocatechin gallate, hydralazine hydrochloride, decitabine, procaine hydrochloride and RG108, in particular decitabine.

### Chemo Drugs

In some embodiments, anticancer compounds may include a chemo drug, in particular selected in a group comprising melphalan, melflufen, cyclophosphamide, doxorubicin, liposomal doxorubicin, vincristine, bendamustine.

### Corticosteroids

In some embodiments, anticancer compounds may include a corticosteroid, in particular selected in a group comprising dexamethasone, methylprednisolone and prednisone.

### Histone Deacetylase (HDAC) Inhibitors

In some embodiments, anticancer compounds may include a histone deacetylase (HDAC) inhibitor, in particular Panobinostat or ricolinostat.

### Therapeutic Monoclonal Antibodies (moAb)

In some embodiments, anticancer compounds may include a monoclonal antibody, in particular selected in a group comprising anti-CD38, anti-SLAMF7 and/or antiBCMA monoclonal antibody. In particular, mention may be made of daratumumab, isatuximab and elotuzumab.

Daratumumab is an IgG1 kappa fully human moAb that targets CD38, which is highly expressed on malignant MM cells. This moAb acts via CDC (complement-dependent cytotoxicity), ADCC, ADCP and immunomodulatory effects due to killing of CD38 positive immune suppressor cells. Daratumumab may be combined with pomalidomide and dexamethasone, or bortezomib and dexamethasone, or carfilzomib and dexamethasone. Isatuximab (SAR650984) is another humanized IgG1 chemeric moAb that targets CD38. Isatuximab may be combined with Pomalidomide and Dexamethasone.

Elotuzumab is a humanized IgG1 monoclonal antibody that targets SALMF7, which is highly expressed on PCs, natural killer cells and monocytes. It induces ADCC while also activating NK cells and inhibiting MM cell adhesion to BMSCs. It may be used in combination with lenalidomide and dexamethasone, or with pomalidomide and dexamethasone.

Monoclonal antibodies have been used to develop Ab drug conjugates (ADC), which are moAbs conjugated to cytotoxic compounds (such as auristatin) via synthetic linkers. As example, mention may be made to a BCMA-aurostatin immunotoxin.

### Bispecific T-Cell Engager (BiTES)

BiTES are bispecific antibodies which bind to specific tumor antigens on one side, and to the CD3 epsilon chain of the T-cell receptor complex on the other. In MM, CD19, CD38, CD138, BCM1, GPRC5D and Fc receptor-like 5 antigens have been tested with early promising responses from BCMA BiTEs treatment in MM. mention may be made to AMG701 that may be combined with lenalidomide or pomalidomide.

### BH3 Mimetics

BH3 mimetics are promising drugs for hematologic malignancies that trigger cell death by promoting the release of proapoptotic BCL2 family members from antiapoptotic proteins. Myeloid cell leukemia-1 (MCL-1) is an anti-apoptotic member of the B-cell lymphoma-2 (BCL-2) family of proteins that regulates apoptosis. AZD5991 is a potent and direct inhibitor of Mcl-1 with high selectivity versus other Bcl-2 family proteins. The compound AZD5991 is under clinical studies (NCT03218683) in MM. BCL2 inhibitors are also used in the treatment of a subset of myeloma characterized by the t(11; 14) translocation.

### CART-T Cells

Cellular therapies represent an optimal strategy to restore host immune surveillance using either adoptive T-cell (ACT) or engineered T cell approaches. Different CAR-T products targeting CD38 and/or BCMA are under studies.

It is within the skills of a physician to determine the specific therapeutically effective dosage regimen, as this dosage regimen will be dependent upon a variety of factors including, but not limited to: the stage of the multiple myeloma and the severity of the disease; the age; the body weight; general health; the sex; the diet; the time course of administration; the route of administration; the duration of the treatment; the drugs that are concomitantly administered in combination with the pharmaceutical composition within the scope of the present invention.

In some embodiments, the dosage regimen of the immunomodulatory agent and/or the compound of formula (I) may range from about 0.0001 mg to about 1,000 mg per adult per day. Preferably, the individual is administered with an amount of about 0.0001, 0.0005, 0.001, 0.005, 0.01, 0.05, 0.1, 0.5, 1.0, 2.5, 5.0, 7.5, 10.0, 15.0, 20.0, 25.0, 50.0, 75.0, 100, 250, 500 and 750 mg of the immunomodulatory agent and/or the salinomycin derivative in order to adjust the dosage regimen that is the most suitable to a particular individual in need of the treatment.

The pharmaceutical composition or pharmaceutical product disclosed herein may be administered by any suitable route, i.e. including, but not limited to, an oral, sublingual, buccal, subcutaneous, transdermal, topical, intraperitoneal, intramuscular, intravenous, subdermal, intrathecal and intranasal and rectal administration.

In a particular embodiment, the combination product comprises a compound of formula (I) as disclosed herein, a chemodrug and an immunomodulatory agent. In particular, the compound of formula (I) is Ironomycin, the chemodrug is Melphalan and the immunomodulatory agent is Pomalidomide or Lenalidomide.

In a particular embodiment, the combination product comprises a compound of formula (I) as disclosed herein, a cortocosteroid and an immunomodulatory agent. In particular, the compound of formula (I) is Ironomycin, the corticosteroid is Dexamethasone and the immunomodulatory agent is Pomalidomide or Lenalidomide.

In a particular embodiment, the combination product comprises a compound of formula (I) as disclosed herein, a monoclonal antibody and an immunomodulatory agent. In particular, the compound of formula (I) is Ironomycin, the monoclonal antibody is Daratumumab and the immunomodulatory agent is Pomalidomide or Lenalidomide.

In some embodiments, the salinomycin derivative of formula (I) used in the present invention may be combined with the following combinations:
melphalan and prednisone;
melphalan, prednisone and thalidomide;
melphalan, prednisone and bortezomib;
vincristine, doxorubicin, and dexamethasone;
thalidomide and dexamethasone;
lenalidomide and dexamethasone;
bortezomib, doxorubicin, and dexamethasone;
bortezomib, dexamethasone, and thalidomide;
bortezomib, dexamethasone, and lenalidomide;
liposomal doxorubicin, vincristine and dexamethasone;
carfilzomib, lenalidomide, and dexamethasone
dexamethasone, cyclophosphamide, etoposide, and cisplatin;
dexamethasone, thalidomide, cisplatin, doxorubicin, cyclophosphamide, and etoposide;
dexamethasone, thalidomide, cisplatin, doxorubicin, cyclophosphamide, and etoposide and bortezomib;
panobinostat, bortezomib, and dexamethasone;
ixazomib, lenalidomide, and dexamethasone;
daratumumab, lenalidomide and dexamethasone;
daratumumab, bortezomib, melphalan, prednisolone;
daratumumab, bortezomib, thalidomide and dexamethasone;
daratumumab, bortezomib and dexamethasone;
daratumumab, carfilzomib and dexamethasone;
and
elotuzumab, lenalidomide, and dexamethasone.

In another particular embodiment, the said pharmaceutical product is used in a method for treating subjects likely to display an MM relapse and/or death, or subjects refractory or resistant to a first line treatment.

### Pharmaceutical Product (as Such)

The invention also relates to a pharmaceutical product comprising:
(i) a compound of formula (I) according to claim 1 wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl,
(ii) an anticancer agent selected in the group consisting of lenalidomide, pomalidomide (immunomodulatory agents), melphalan (chemo-drug), carfilzomib (proteasome inhibitor), AZD-5991 (MCL1 inhibitor), and combinations thereof and (iii) optionally another anticancer agent or cellular therapy for treating MM selected in the group consisting of proteasome inhibitors (Pis), immunomodulatory agents, chemo drugs, inhibitors of nuclear export in particular exportin 1 inhibitor, corticosteroids, histone deacetylase (HDAC) inhibitors, therapeutic monoclonal antibodies (moAb) in particular anti-CD38, anti-SLAMF7, and/or anti BCMA, antibodies drug conjugates (ADC), bispecific T-cell engager (BiTES), MCL1 inhibitors and other BH3 mimetics, CART-T cells and combinations thereof.

Examples of such compounds are disclosed above.

In a particular embodiment:
(i) the compound of formula (I) is such as W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl, and
(ii) the anticancer agent is selected in the group consisting of lenalidomide, pomalidomide, melphalan, and a combination thereof.

In a particular embodiment, the invention concerns the pharmaceutical product as disclosed above for use in a method for treating subjects likely to display an MM relapse and/or death, or subjects refractory or resistant to a first line treatment.

The invention also relates to a pharmaceutical composition as defined above or a pharmaceutical product as defined above for use in the treatment of a MM subject that has been identified as having a poor outcome by a method comprising the steps of:
a) Measuring the expression level of at least 3, in particular at least 5 genes and/or proteins encoded by the said at least 3, in particular the said at least 5 genes selected in the group consisting of CYBRD1, EPAS1, FBXL5, PPOX, SLC39A14 and STEAP1 involved in the iron metabolism, in a biological sample obtained from said subject;
b) Calculating a score value from said expression level obtained at step a)
c) Classifying and identifying the said subject as having a poor outcome according to the score value in comparison to a predetermined reference value (PRV).

The invention also relates to a method for treating a multiple myeloma subject in need thereof, comprising the administration of a compound of formula (I) as disclosed herein, optionally in a combination with an anticancer treatment, especially in a combination with an anti-MM treatment, which includes a treatment with one more other anti-MM active ingredients, as disclosed above.

This invention also pertains to a method for treating a multiple myeloma subject in need thereof comprising the steps of:
A) performing a method predicting the likelihood of the said subject having multiple myeloma to respond to a therapeutic treatment comprising a compound of formula (I) as disclosed herein, comprising the steps of:
a) measuring the expression level of at least 3, in particular at least 5 genes and/or proteins encoded by the said at least 3, in particular the said at least 5 genes selected in the group consisting of CYBRD1, EPAS1, FBXL5, PPOX, SLC39A14 and STEAP1 involved in the iron metabolism, in a biological sample obtained from said subject;
b) calculating a score value from said expression level obtained at step a);
c) classifying and identifying the said subject as having a poor outcome according to the score value in comparison to a predetermined reference value (PRV),
B) if the said subject has been classified as having a poor outcome at step A), then administering to the said subject a compound of formula (I), preferably in combination with one or more other anti-MM active ingredients, such as one or more immunomodulatory agent.

### In Vitro Method for Identifying MM Subject with Poor Outcome

The present invention also concerns an in vitro method for identifying MM subject with a poor outcome that may benefit from a therapeutic treatment comprising a compound of formula (I), enantiomers, mixture of enantiomers, diastereoisomers and mixture of diastereoisomers thereof as defined in the present invention or a pharmaceutical composition as defined in the invention or a pharmaceutical product as defined in the invention, comprising the steps of:
a) Measuring the expression level of at least 3, in particular at least 5 genes and/or proteins encoded by the said at least 3, in particular the said at least 5 genes selected in the group consisting of CYBRD1, EPAS1, FBXL5, PPOX, SLC39A14, and STEAP1 involved in the iron metabolism, in a biological sample obtained from said subject;
b) Calculating a score value from said expression level obtained at step a)
c) Classifying and identifying the said subject as having a poor outcome according to the score value in comparison to a predetermined reference value (PRV).

The in vitro methods of the present invention optionally comprise one or more housekeeping gene(s) for normalization of the data.

By “housekeeping genes”, it is meant genes that are constitutively expressed at a relatively constant level across many or all known conditions, because they code for proteins that are constantly required by the cell, hence, they are essential to a cell and always present under any conditions. It is assumed that their expression is unaffected by experimental conditions. The proteins they code are generally involved in the basic functions necessary for the sustenance or maintenance of the cell. Non-limitating examples of housekeeping genes that may be used in methods of the invention include:
HPRT1 (hypoxanthine phosphoribosyltransferase 1),
UBC (ubiquitin C),
YWHAZ (tyrosine 3-monooxygenase/tryptophan 5-monooxygenase activation protein, zeta polypeptide),
B2M (beta-2-microglobulin),
GAPDH (glyceraldehyde-3-phosphate dehydrogenase),
FPGS (folylpolyglutamate synthase),
DECR1 (2,4-dienoyl CoA reductase 1, mitochondrial),
PPIB (peptidylprolyl isomerase B (cyclophilin B)),
ACTB (actin P),
PSMB2 (proteasome (prosome, macropain) subunit, beta type, 2),
GPS1 (G protein pathway suppressor 1),
CANX (calnexin),
NACA (nascent polypeptide-associated complex alpha subunit),
TAX1 BP1 (Tax1 (human T-cell leukemia virus type 1) binding protein 1), and
PSMD2 (proteasome (prosome, macropain) 26S subunit, non-ATPase, 2).

When such housekeeping genes are added to the expression profile (it is not always necessary), they are used for normalization purpose. In this case, the number of housekeeping genes used for normalization in methods according to the invention is preferably comprised between one and five with a preference for three.

The in vitro methods of the present invention comprise a step of measuring the expression level of at least 2, 3, 4, 5, 6 genes useful for the outcome prognostic, also named ‘prognosis genes or genes of interest’ according to the invention.

The present invention also relates to a kit dedicated to in vitro methods according to the invention, in particular for MM subjects, comprising or consisting of reagents for determining the expression level of at least 2, preferably at least 5 genes and/or proteins selected in the group consisting of CYBRD1, EPAS1, FBXL5, PPOX, SLC39A14, and STEAP1 in a sample of said subject.

The present invention also relates to systems (and computer readable medium for causing computer systems) to perform the in vitro methods of the invention, based on above-described expression levels of genes and/or proteins as identified above.

In particular, the system includes a machine-readable memory, such as a computer or/and a calculator, and a processor configured to compute R Maxstat function and Cox multivariate function, according to the invention. This system is dedicated to perform the in vitro methods according to the invention in particular for identifying MM subjects with a poor outcome.

In particular, the system 1 for analyzing a biological sample of a subject affected by MM, comprises:
(a) a determination module 2 configured to receive a biological sample and to determine expression level information concerning the prognosis genes as disclosed in the present invention and optionally one or more housekeeping gene(s);
(b) a storage device 3 configured to store the expression level information from the determination module;
(c) a comparison module 4 adapted to compare the expression level information stored on the storage device with reference data, and to provide a comparison result, wherein the comparison result is indicative of the outcome of the subject; and
(d) a display module 5 for displaying a content based in part on the comparison result for the user, wherein the content is a signal indicative of the outcome of the subject.

By ‘at least 2, in particular at least 5’ genes and/or proteins, it means 2, 3, in particular 4, 5, 6, genes and/or proteins.

In an embodiment, the combination of 2 genes and/or proteins encoded by the said genes selected in the group consisting of CYBRD1, EPAS1, FBXL5, PPOX, SLC39A14, STEAP1 is evaluated.

In an embodiment, the combination of 3 genes and/or proteins encoded by the said genes selected in the group consisting of CYBRD1, EPAS1, FBXL5, PPOX, SLC39A14, and STEAP1 is evaluated.

In an embodiment, the combination of 4 genes and/or proteins encoded by the said genes selected in the group consisting of CYBRD1, EPAS1, FBXL5, PPOX, SLC39A14 and STEAP1 is evaluated.

In an embodiment, the combination of 5 genes and/or proteins encoded by the said genes selected in the group consisting of CYBRD1, EPAS1, FBXL5, PPOX, SLC39A14, and STEAP1, is evaluated.

In another embodiment, the combination of 6 genes and/or proteins encoded by the said genes selected in the group consisting of CYBRD1, EPAS1, FBXL5, PPOX, SLC39A14, and STEAP1, is evaluated.

The NCBI references for each gene are mentioned in the table 1 hereunder:

TABLE 1

| | | Ref Seq | Ref Seq | |
|---|---|---|---|---|
| Gene | | Transcript ID | Protein ID | Gene |
| symbol | Gene Name | (NCBI) | (NCBI) | ID |
| CYBRD1 | cytochrome b | NM_024843.4 | NP_079119 | 79901 |
| | reductase 1 | NM_001127383 | NP_001120855 | |
| | | NM_001256909 | NP_001243838 | |
| EPAS1 | endothelial PAS | NM_001430 | NP_001421 | 2034 |
| | domain protein 1 | | | |
| FBXL5 | F-box and | NM_001193534 | NP_001180463 | 26234 |
| | leucine-rich | NM_001193535 | NP_001180464 | |
| | repeat protein 5 | NM_012161 | NP_036293 | |
| PPOX | protoporphyrinogen | NM_000309 | NP_000300 | 27005 |
| | oxidase | NM_001122764 | NP_001116236 | |
| SLC39A14 | solute carrier | NM_001128431 | NP_001121903 | 23516 |
| | family 39 (zinc | NM_001135153 | NP_001128625 | |
| | transporter), | NM_015359 | NP_001128626 | |
| | member 14 | | NP_056174 | |
| STEAP1 | Six transmembrane | NM_012449 | NP_036581 | 26872 |
| | epithelial antigen | | | |
| | of the prostate 1 | | | |

### Expression Level of the Set of the Said Genes or Proteins of Interest (‘Prognosis Genes’)

Such measures are made in vitro, starting from a subject's sample, and necessary involve transformation of the sample. Indeed, no measure of a specific gene expression level can be made without some type of transformation of the sample. Most technologies rely on the use of reagents specifically binding to the RNA of interest, thus resulting in a modified sample further including the detection reagent. In addition, most technologies also involve some preliminary extraction of RNA from the subject's sample before binding to a specific reagent. The claimed method may thus also comprise a preliminary step of extracting RNA from the subject's sample.

The expression level of the set of genes and/or proteins, in particular selected in the group consisting of CYBRD1, EPAS1, FBXL5, PPOX, SLC39A14, and STEAP1, according to the invention, may be measured by any techniques commonly used.

The presence or level of said genes is determined by usual method known from man skilled in the art. In particular, each gene expression level may be measured at the genomic and/or nucleic and/or protein level. In a preferred embodiment, the expression profile is determined by measuring the amount of nucleic acid transcripts of each gene, such as PCR, quantitative PCR (qPCR), NGS (Next-Generation Sequencing (NGS)) and RNA sequencing. In another embodiment, the expression profile is determined by measuring the amount of protein produced by each of the genes.

The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, next generation sequencing and hybridization with a labelled probe. PCR primers for the DNA amplicons encompassing the genes of interest disclosed above were designed using the genomic sequence obtained from the NCBI.

In particular, the level of mRNA expression for each of the genes of the set may be performed by the well-known techniques of the skilled in the art such as hybridization technique and/or amplification technique (PCR), using suitable primers or probes that are specific for each of the genes mRNA.

Illustratively, mRNA may be extracted, for example using lytic enzymes or chemical solutions or extracted by commercially available nucleic-acid-binding resins following the manufacturer's instructions. Extracted mRNA may be subsequently detected by hybridization, such as Northern blot, and/or amplification, such as quantitative or semiquantitative RT-PCR. Other methods of amplification include ligase chain reaction (LCR), transcription-mediated amplification (TMA), strand displacement amplification (SDA) and nucleic acid sequence based amplification (NASBA). In some embodiments, the level of mRNA expression for each of the genes of interest may be measured by the mean of quantification of the cDNA synthesized from said mRNA, as a template, by one reverse transcriptase.

The amount of mRNA can be measured by any technology known by a person skilled in the art, including mRNA microarrays, quantitative PCR, next generation sequencing and hybridization with a labelled probe. In particular, real time quantitative RT-PCR (qRT-PCR) may be useful. In some embodiments, qRT-PCR can be used for both the detection and quantification of RNA targets. Commercially available qRT-PCR based methods (e.g., Taqman® Array) may for instance be employed, the design of primers and/or probe being easily made based on the sequence of ‘prognostic genes’ disclosed above. mRNA assays or arrays can also be used to assess the levels of the mRNAs in a sample. In some embodiments, mRNA oligonucleotide array can be prepared or purchased. An array typically contains a solid support and at least one oligonucleotide contacting the support, where the oligonucleotide corresponds to at least a portion of a mRNA.

Any suitable assay platform can be used to determine the presence of the mRNA in a sample. For example, an assay may be in the form of a membrane, a chip, a disk, a test strip, a filter, a microsphere, a multiwell plate, and the like. An assay system may have a solid support on which an oligonucleotide corresponding to the mRNA is attached. The solid support may comprise, for example, a plastic, silicon, a metal, a resin, or a glass. The assay components can be prepared and packaged together as a kit for detecting an mRNA. To determine the expression profile of a target nucleic sample, said sample is labelled, contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the person skilled in the art.

Methods for determining the quantity of mRNA by microarrays or by RNA sequencing may also be used. In certain embodiments, complexes between the double-stranded nucleic acids resulting from amplification and fluorescent SYBR® molecules may be obtained and then the fluorescence signal generated by the SYBR® molecules complexed with the said amplified nucleic acids may be measured. Identification of suitable primers that are specific for each of the genes mRNA consists of a routine work for the one skilled in the art.

In a particular embodiment and as illustrated in the examples for MM subjects, the method for determining the quantity of mRNA by microarrays uses probesets for the specific 6 prognostic genes disclosed above. Mention may be made of the Affymetrix HG-U133 plus 2.0 microarrays and probesets ID related to said specific 6 prognostic genes. In a particular embodiment, method for determining the quantity of mRNA by microarrays uses 6 probesets for the specific 6 prognostic genes, as illustrated in the further examples.

In some embodiments, detection by hybridization may be performed with a detectable lable, such as fluorescent probes, enzymatic reactions or other ligands (eg avidin/biotin).

The presence or level of said proteins may be measured by well-known techniques including detection and quantification of the protein of interest by the means of any type of ligand molecule that specifically binds thereto, including nucleic acids (for example nucleic acids selected for binding through the well-known SELEX method), antibodies and antibody fragments. The antibodies to said given protein of interest may be easily obtained with the conventional techniques, including generation of antibody-producing hybridomas.

Thus, in preferred embodiments, expression of a marker is assessed using for example:
a radio-labelled antibody, in particular, a radioactive moiety suitable for the invention may for example be selected within the group comprising 3H, 1211, 1231, 14C or 32P;
a chromophore-labelled or a fluorophore-labelled antibody, wherein a luminescent marker, and in particular a fluorescent marker, suitable for the invention may be any marker commonly used in the field such as fluorescein, fluorescent probes, coumarin and its derivatives, phycoerythrin and its derivatives, or fluorescent proteins such as GFP or the DsRed;
a polymer-backbone-antibody;
an enzyme-labelled antibody, said labelling enzyme suitable for the invention may be an alkaline phosphatase, a tyrosinase, a peroxydase, or a glucosidase; for example, suitable avidin-labelled enzyme may be an avidin-Horse Radish Peroxydase (HRP), and a suitable substrate may be AEC, 5-bromo-4-chloro-3-indolyl phosphate (BCIP), nitro blue tetrazolium chloride (NBT);
an antibody derivative, for example an antibody conjugated with a substrate or with the protein or ligand of a protein-ligand pair, in particular a biotin, a streptavidin or an antibody binding the polyhistidine tag;
an antibody fragment, for example a single-chain antibody, an isolated antibody hypervariable domain, etc., which binds specifically to a marker protein or a fragment thereof, including a marker protein which has undergone all or a portion of its normal post-translational modification.

In a particular and preferred embodiment, expression of a marker is assessed using a GFP fluorescent protein.

In vitro techniques for detection of a biological marker protein include enzyme linked immunosorbent assays (ELISAs), Western blots, immunoprecipitations and immunofluorescence.

In a particular and preferred embodiment, the preferred in vitro methods for detecting and quantifying level expression of said genes of interest according to the present invention, include micro-arrays, NGS, RNA sequencing and PCR techniques.

### Calculation of a Score Value (‘Iron Score’) from Said Expression Level of Genes or Proteins of Interest

The score value or ‘prognosis score’ or ‘iron score’ according to the invention, based on the expression level of the ‘prognosis genes’ as defined above, will help classifying the MM subjects as having a ‘good outcome’ or a ‘bad outcome’.

The lower the expression of genes related to ‘bad outcome’ is, the better for the subject's survival. Therefore, the higher the level of iron score is, the more likely the subject is to respond to a treatment targeting iron metabolism. In a preferred embodiment, the subject may thus be predicted as having ‘poor outcome’ and consequently being likely to respond to a treatment targeting iron metabolism based on comparison of the expression level of said prognosis genes in the patient's sample with one or more threshold value(s) (predetermined reference value, PREV).

In a particular embodiment, the patient is considered as having poor outcome, when the iron score is higher than a threshold value. Such a threshold value may be determined based on a pool of reference samples, as defined above. In this embodiment, patients are classified into two groups based on said expression level of prognosis genes, depending if this expression level is lower or greater than said threshold value. Patients with iron score higher than the threshold value are considered as having a poor outcome and likely to respond to treatment targeting iron metabolism.

In another embodiment, the method further comprises determining a prognostic score based on the expression level of said prognosis genes, wherein the prognostic score indicates whether the patient has a poor outcome. In particular, said prognosis score may indicate whether the patient is likely to have a poor outcome or a bad outcome if it is higher or lower than a predetermined threshold value (PREV or PREL) (dichotomized result).

As a result, a prognosis score may be determined based on the analysis of the correlation between the expression level of said prognosis genes of the invention and progression free survival (PFS) or overall survival (OS) of a pool of reference samples, as defined above. A PFS and/or OS score, which is a function correlating PFS or OS to the expression level of said prognosis genes of the invention, may thus be used as prognosis score for prediction of the outcome of the subject.

The expression level for each combination of the 6 genes and/or proteins of interest as disclosed above according to the invention, may be associated with a score value, also named ‘iron-score’ in the present invention.

Following the measurement of the expression level of at least 2, in particular at least 5 or more genes and/or proteins encoded by the said 5 or more genes selected in a group consisting of CYBRD1, EPAS1, FBXL5, PPOX, SLC39A14, and STEAP1, in a biological sample obtained from a MM subject (step a) of the method), the computation of a score value may be performed by a method comprising the following steps:
i) comparing the expression level determined at step a) with a predetermined reference expression level (PREL);
ii) calculating the score value (‘iron score’) with the following formula:

$$Score = {\sum }_{i = 1}^{n} {β}_{i} \times {C}_{i}$$

wherein
n represents the number of genes and/or protein which expression level is measured, i.e. n being comprised from 1 to 6, in particular from 3 to 6,
βi represents the regression β coefficient reference value for a given gene or protein, and
Ci represents “1” if the expression level of said gene or protein is higher than the predetermined reference level (PREL) or Ci represents “−1” if the expression level of the gene or the protein is lower than or equal to the predetermined reference level (PREL).

The predetermined reference level (PREL) is often referred as to “maxstat value” or “maxstat cutpoint”.

In some embodiments, a good prognosis status or ‘good outcome’ refers to an individual having a score value lower than or equal to a predetermined reference value (PRV).

In some embodiments, a bad prognosis status or ‘bad outcome’ refers to an individual having a score value higher than a predetermined reference value (PRV).

The “regression β coefficient reference value” may be easily determined by the skilled man in the art for each gene or protein using the well-known statistical Cox model, which is based on a modelling approach to analyze survival data. The purpose of the model is to simultaneously explore the effects of several variables on survival. When it is used to analyze the survival of patients in a clinical trial, the model allows isolating the effects of the treatment from the effects of other variables. The Cox model may also be referred as to proportional hazards regression analysis. In particular, this model is a regression analysis of the survival times (or more specifically, the so-called “hazard function”) with respect to defined variables. The “hazard function” is the probability that an individual will experience an event, e.g. death, within a small time interval, given that the individual has survived up to the beginning of the interval. It can therefore be interpreted as the risk of dying at time t. The quantity h0 (t) is the baseline or underlying hazard function and corresponds to the probability of dying (or reaching an event) when all the defined variables are zero. The baseline hazard function is analogous to the intercept in ordinary regression (since exp0=1). The “regression coefficient β” gives the proportional change that can be expected in the hazard, related to changes in the defined variables. The coefficient β is estimated by a statistical method called maximum likelihood. In survival analysis, the hazard ratio (HR) (Hazard Ratio=exp(β)) is the ratio of the hazard rates corresponding to the conditions described by two sets of defined variables.

Predetermined reference values, such as PREL or PRV, which are used for comparison purposes may consist of “cut-off” values.

For example, each reference (“cut-off”) value PREL for each gene or protein may be determined by carrying out a method comprising the following steps:
a) providing a collection of samples from subjects (patients) suffering from MM (‘reference samples’);
b) determining the expression level of the relevant gene or protein for each sample contained in the collection provided at step a);
c) ranking the samples according to said expression level;
d) classifying said samples in pairs of subsets of increasing, respectively decreasing, number of members ranked according to their expression level;
e) providing, for each sample provided at step a), information relating to the actual clinical outcome for the corresponding MM patient (i.e., the duration of the disease-free survival (DFS), or the event free survival (EFS) or the overall survival (OS) or both);
f) for each pair of subsets of tumor tissue samples, obtaining a Kaplan Meier percentage of survival curve;
g) for each pair of subsets of tumor tissue samples calculating the statistical significance (p value) between both subsets;
h) selecting as reference value PREL for the expression level, the value of expression level for which the p value is the smallest.

As an illustration, the expression level of a gene or a protein of interest may be assessed for 100 samples (‘reference samples’) of 100 subjects (patients). The 100 samples are ranked according to the expression level of said given gene or protein. Sample 1 may have the highest expression level and sample 100 may have the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding MM patient, Kaplan Meier curves may be prepared for each of the 99 groups of two subsets. Also, for each of the 99 groups, the p value between both subsets was calculated. The reference value PREL is then selected such as the discrimination based on the criterion of the minimum p value is the strongest. In other words, the expression level corresponding to the boundary between both subsets for which the p value is minimum is considered as the reference value. It should be noted that according to the experiments made by the inventors, the reference value PREL is not necessarily the median value of expression levels.

The skilled in the art also understands that the same technique of assessment of the PRV could be used for obtaining the reference value and thereafter for assessment of the response to the targeted treatment of the present invention comprising an inhibitor of iron metabolism. However in one embodiment, the reference value PRV is the median value of PRV.

As illustrated further in the examples of the invention, the prognostic information of these 6 genes of interest (‘prognosis genes’) was then combined in a GEP (Gene Expression Profile)-based iron-score. The ‘iron score’ is defined by the sum of the beta coefficients of the Cox model for each prognostic gene, weighted by ±1 according to the patient signal above or below the probe set Maxstat value as previously described (Herviou et al., 2018). Maxstat algorithm segregated the TT2 cohort into two groups with 23.8% of the patients with an iron score >−0.012126 and 76.2% of the patients with an iron score ≤−0.012126 with a maximum difference in overall survival (OS). Patients with high-risk iron score have a median OS of around 40 months versus not reached for patients with low iron score (P=2.73 0.10⁻¹⁵)) in the TT2 cohort. As illustrated in the examples, the prognostic value of the iron score was validated in the three additional independent cohorts for OS.

Altogether, these data highlight a deregulation of iron metabolism genes in association with a poor outcome in MM.

In particular embodiment, the regression β coefficient reference value, the hazard ratio and the reference value PREP for each of the 6 genes or proteins of interest were measured. These values were measured on references samples of MM subjects (>200 samples) but may vary from 5 to 15% depending of the number of reference samples.

The highest the number of reference samples, the better for the reliability of the method of prediction of the outcome of the subject tested according to the invention.

The Table 2 below illustrates relevant parameter ranges for Maxstat_Cutpoint, beta coefficient and Hazard ratio (HR) for each of the 6 genes of interest.

TABLE 2

| | | | | | Muktiple | Hazard_Ratio | | |
|---|---|---|---|---|---|---|---|---|
| ID | Name | Maxstat_Cutpoint | Chisq | pvalue | testing | (HR) | expB | Prognostic |
| 222453_at | CYBRD1 | 11 | 9.8 | 0.0018 | 1.13E−02 | 0.55 | −0.2596373 | Good |
| 200878_at | EPAS1 | 108 | 22 | 2.70E−06 | 1.70E−04 | 0.37 | −0.4317983 | Good |
| 209004_s_at | FBXL5 | 3611 | 10 | 0.0016 | 1.12E−02 | 0.55 | −0.2596373 | Good |
| 204788_s_at | PPOX | 990 | 15 | 0.00012 | 2.52E−03 | 2.1 | 0.32221929 | Bad |
| 212110_at | SLC39A14 | 4060 | 13 | 0.00026 | 2.73E−03 | 0.47 | −0.3279021 | Good |
| 205542_at | STEAP1 | 325 | 6.1 | 0.013 | 4.55E−02 | 1.7 | 0.23044892 | Bad |

This table 2 and related FIG. 2 show that the genes PPOX and STEAP1 have the higher Hazard ratio (HR>2), meaning that an iron score based on the expression level of at least these genes would be a good prognostic marker for MM patients having poor outcome.

The score may be generated by a computer program and may be used in the in vitro method according to the invention in particular for identifying a MM subject with a poor outcome that may benefit of a targeted treatment comprising an inhibitor of iron metabolism, and/or for further monitoring the efficacy of a targeted therapeutic treatment.

So, the in vitro method for identifying MM subject with a poor outcome that may benefit from a therapeutic treatment according to the invention, as defined above, comprises the steps of:
a) Measuring the expression level of at least 3, in particular at least 5 genes and/or proteins encoded by the said at least 3, in particular the said at least 5 genes selected in the group consisting of CYBRD1, EPAS1, FBXL5, PPOX, SLC39A14 and STEAP1 involved in the iron metabolism, in a biological sample obtained from said subject;
b) Calculating a score value from said expression level obtained at step a)
c) Classifying and identifying the said subject as having a poor outcome according to the score value in comparison to a predetermined reference value (PRV).

The expression level of the said genes or proteins of interest at step a) are measured according to the detection and/or quantification methods well known in the art. Examples of such methods are disclosed above.

The calculation of the score value (‘iron score’) at step b) is made as disclosed above, in particular by:
i) comparing the expression level determined at step a) with a predetermined reference expression level (PREL);
ii) calculating the score value with the following formula:

$$Score = {\sum }_{i = 1}^{n} {β}_{i} \times {C}_{i}$$

wherein
n represents the number of genes and/or protein which expression level is measured, i.e. n being comprised from 3 to 6,
βi represents the regression β coefficient reference value for a given gene or protein, and
Ci represents “1” if the expression level of said gene or protein is higher than the predetermined reference level (PREL) or Ci represents “−1” if the expression level of the gene or the protein is lower than or equal to the predetermined reference level (PREL).

The classification of the subject according to ‘good outcome’ subgroup and ‘bad outcome’ subgroup is based according to its iron-score value in comparison to a predetermined reference value (PRV).

In the present invention, a subject with a ‘poor outcome’ refers to an individual having a score value higher than a predetermined reference value (PRV).

In a particular embodiment, for MM subjects, when the iron score is based on the expression level of the 6 genes or proteins consisting of CYBRD1, EPAS1, FBXL5, PPOX, SLC39A14, and STEAP1, the predetermined reference value (PRV) or ‘cutpoint’ is −0.012126, meaning that in the step c) of the in vitro method described above, the subject with a poor outcome according to the iron score are the ones having an iron score value higher than −0.012126.

### Method for Monitoring the Efficacy of a Targeted Therapeutic Treatment

Another object of the invention is an in vitro method for monitoring the efficacy of a therapeutic treatment targeting iron metabolism in a subject having a MM and undergoing said treatment, comprising the steps of:
a) Measuring the expression level of at least 2, in particular at least 5 genes and/or proteins encoded by the said at least 2, preferably at least 5 genes selected in the group consisting of CYBRD1, EPAS1, FBXL5, PPOX, SLC39A14, and STEAP1 involved in the iron metabolism, in a biological sample obtained from said subject at a time T1 before or during or after the subject has been administered said therapeutic treatment targeting iron metabolism;
b) Calculating a first score value at time T1 from said expression level obtained at step a),
c) Measuring the expression level of at least 2, in particular at least 5 genes and/or proteins encoded by the said at least 2, preferably at least 5 genes selected in the group consisting of CYBRD1, EPAS1, FBXL5, PPOX, SLC39A14, and STEAP1 involved in the iron metabolism, in a biological sample obtained from said subject at a time T2 before or during or after the subject has been administered the said therapeutic treatment targeting iron metabolism, wherein said time T2 is posterior to said time T1;
d) Calculating a second score value at time T2 from said expression level obtained at step c),
e) Assessing the efficacy of a therapeutic treatment based on the comparison the second score value at T2 obtained at step d) with the first score value at T1 obtained at step b).

The expression level of genes or proteins of interest according to the invention at step a) and d) are made as disclosed above.

The first and second score values (iron-score values), respectively at time T1 and time T2, are made as disclosed above.

In a preferred embodiment, the invention concerns an in vitro method for monitoring the efficacy of a therapeutic treatment targeting iron metabolism in a subject having MM and undergoing said treatment, comprising the steps of:
a) Measuring the expression level of the 11 genes or proteins consisting of CYBRD1, EPAS1, FBXL5, PPOX, SLC39A14, and STEAP1 involved in the iron metabolism, in a biological sample obtained from said subject at a time T1 before the subject has been administered said therapeutic treatment comprising an active agent against MM and/or an inhibitor of iron metabolism;
b) Calculating a first score value at time T1 from said expression level obtained at step a),
c) Measuring the expression level of the 11 genes or proteins consisting of CYBRD1, EPAS1, FBXL5, PPOX, SLC39A14, and STEAP1 involved in the iron metabolism, in a biological sample obtained from said subject at a time T2 after the subject has been administered the said therapeutic treatment comprising an active agent against MM and/or an inhibitor of iron metabolism, wherein said time T2 is posterior to said time T1;
d) Calculating a second score value at time T2 from said expression level obtained at step c),
e) Assessing the efficacy of a therapeutic treatment based on the comparison the second score value at T2 obtained at step d) with the first score value at T1 obtained at step b).

### Kits Dedicated for In Vitro Methods of the Invention

The kits of the invention are dedicated for in vitro methods of the invention.

By “dedicated”, it is meant that reagents for the determination of an expression level of genes and/or proteins as identified above in the kit of the invention essentially consist of reagents for determining the expression level of the above (i) expression profiles, optionally with one or more housekeeping gene(s), and thus comprise a minimum of reagents for determining the expression of other genes than those mentioned in above described (i) expression profiles and housekeeping genes. For instance, a dedicated kit of the invention preferably comprises no more than 20, preferably no more than 12, preferably no more than 10, preferably no more than 9, 8, 7, 6, 5, 4, 3, 2, or 1 reagent(s) for determining the expression level of a gene that does not belong to one of the above described (i) expression profiles and that is not a housekeeping gene.

Such a kit may further comprise instructions for determination of poor or good outcome of the subject.

So the present invention relates to a kit dedicated to in vitro methods of the invention, in particular for determining whether a MM subject has a high risk of death and/or relapse, comprising or consisting of reagents for determining the expression level of at least 2, preferably at least 5 genes and/or proteins selected in the group consisting of CYBRD1, EPAS1, FBXL5, PPOX, SLC39A14, and STEAP1 in a sample of said subject, and no more than 20, preferably no more than 12, preferably no more than 10, preferably no more than 9, 8, 7, 6, 5, 4, 3, 2, or 1 reagent(s) for determining the expression level of a gene that does not belong to one of the above described.

Reagents for determining the expression level of said prognostic genes in a sample of said subject, may notably comprise or consist of primers pairs (forward and reverse primers) and/or probes (in particular labeled probes, comprising a nucleic acid specific for the target sequence and a label attached thereto, in particular a fluorescent label) specific for said prognostic genes or a microarray comprising a sequence specific for said prognostic genes. The design of primers and/or probe can be easily made by those skilled in the art based on the sequences of said genes disclosed above.

In a particular embodiment, said kits comprise specific amplification primers and/or probes for the specific quantitative amplification of transcripts of ‘prognosis genes’ identified above and/or a nucleic microarray for the detection of said ‘prognosis genes’ identified above.

The present invention also relates to a kit dedicated to in vitro methods of the present invention comprising a set of primers and/or probes for measuring the expression level of at the least 2, preferably at least 5 genes and/or proteins encoded by the said at least 2, preferably at least 5 genes selected in the group consisting of CYBRD1, EPAS1, FBXL5, PPOX, SLC39A14, and STEAP1, as a set of prognostic markers for performing an in vitro method as disclosed above. In particular, the said kit comprises no more than 20, preferably no more than 12, preferably no more than 10, preferably no more than 9, 8, 7, 6, 5, 4, 3, 2, or 1 reagent(s) for determining the expression level of a gene that does not belong to one of the above described.

In a first embodiment, the kit of the present invention is used for performing an in vitro method for identifying a MM subject with a poor outcome that may benefit from a targeted therapeutic treatment as disclosed above.

In another embodiment, the kit of the present invention is used for performing an in vitro method for monitoring the efficacy of a therapeutic treatment targeting iron metabolism in a subject having MM and undergoing said treatment.

The kits for detection of poor outcome MM patients or respectively for monitoring the efficacy of a targeted therapeutic treatment, may also comprises all reagents needed for the detection and/or quantification of expression of the said genes or proteins of interest according to the invention.

In a particular embodiment, the kit dedicated to MM subjects comprises a set of probe sets for measuring the expression level of 6 genes and/or proteins encoded by the said 6 genes selected in the group consisting of CYBRD1, EPAS1, FBXL5, PPOX, SLC39A14 and STEAP1. In particular, the said kit comprises no more than 20, preferably no more than 12, preferably no more than 10, preferably no more than 9, 8, 7, 6, 5, 4, 3, 2, or 1 reagent(s) for determining the expression level of a gene that does not belong to one of the above described. The kit may also comprise generic reagents useful for the determination of the expression level of any gene, such as Taq polymerase or an amplification buffer.

The present invention will be now illustrated by the non-limitative examples.

### EXAMPLES

### Material and Methods

### Gene Expression Data Analyses and Building of the Iron Score

Gene expression microarray data from 4 independent cohorts of patients diagnosed with MM patients were used (TT2 cohort (GSE2658)=345; HM cohort (E-MTAB-362)=206; TT3 cohort (E-TABM-1138)=158 and Mulligan cohort (GSE9782)=188 patients at relapse treated with bortezomib monotherapy). These 4 cohorts were respectively referred to in the following publications: TT2 cohort in Barlogie B, Tricot G, Rasmussen E, Anaissie E, van Rhee F, Zangari M et al. (2006); HM cohort in Hose, Dirk et al. (2011); TT3 cohort in Pineda-Roman M, et al. (2008) and Mulligan cohort in Mulligan, G. et al. (2007).

A list of 63 genes related to iron metabolism in cancer was defined, based on Miller et al., 2011 as disclosed hereunder in Table 3.

TABLE 3

| Gene Symbol | Gene Title |
|---|---|
| FTH1 | ferritin, heavy polypeptide 1 |
| EPAS1 | endothelial PAS domain protein 1 |
| HIF1A | hypoxia inducible factor 1, alpha subunit |
| | (basic helix-loop-helix transcription factor) |
| LTF | Lactotransferrin |
| UROS | uroporphyrinogen III synthase |
| HMBS | hydroxymethylbilane synthase |
| FECH | Ferrochelatase |
| SLC11A2 | solute carrier family 11 (proton-coupled |
| | divalent metal ion transporter), member 2 |
| ABCB6 | ATP binding cassette subfamily B member 6 |
| | (Langereis blood group) |
| HMOX1 | heme oxygenase 1 |
| HEPH | Hephaestin |
| PPOX | protoporphyrinogen oxidase |
| CP | ceruloplasmin (ferroxidase) |
| MTF1 | metal-regulatory transcription factor 1 |
| STEAP1 | six transmembrane epithelial antigen of |
| | the prostate 1 |
| FXN | Frataxin |
| ALAS1 | 5′-aminolevulinate synthase 1 |
| LRP2 | LDL receptor related protein 2 |
| ACO1 | aconitase 1, soluble |
| HP /// HPR | haptoglobin /// haptoglobin-related protein |
| TFRC | transferrin receptor |
| UROD | uroporphyrinogen decarboxylase |
| FBXL5 | F-box and leucine-rich repeat protein 5 |
| ISCU | iron-sulfur cluster assembly enzyme |
| ISCA1 | iron-sulfur cluster assembly 1 |
| ABCG2 | ATP binding cassette subfamily G member 2 |
| | (Junior blood group) |
| TFR2 | transferrin receptor 2 |
| HFE | Hemochromatosis |
| SLC39A14 | solute carrier family 39 (zinc transporter), |
| | member 14 |
| LCN2 | lipocalin 2 |
| FTL | ferritin, light polypeptide |
| TMPRSS6 | transmembrane protease, serine 6 |
| CIAO1 | cytosolic iron-sulfur assembly component 1 |
| HMOX2 | heme oxygenase 2 |
| STEAP3 | STEAP family member 3, metalloreductase |
| NFS1 | NFS1 cysteine desulfurase |
| ALAD | aminolevulinate dehydratase |
| SLC22A17 | solute carrier family 22, member 17 |
| NARFL | nuclear prelamin A recognition factor-like |
| NFU1 | NFU1 iron-sulfur cluster scaffold |
| HAMP | hepcidin antimicrobial peptide |
| SFXN3 | sideroflexin 3 |
| CYBRD1 | cytochrome b reductase 1 |
| SLC25A37 | solute carrier family 25 (mitochondrial |
| | iron transporter), member 37 |
| FLVCR1 | feline leukemia virus subgroup C cellular receptor 1 |
| SLC40A1 | solute carrier family 40 (iron-regulated |
| | transporter), member 1 |
| SLC25A28 | solute carrier family 25 (mitochondrial iron |
| | transporter), member 28 |
| MON1A | MON1 secretory trafficking family member A |
| SFXN4 | sideroflexin 4 |
| STEAP2 | STEAP family member 2, metalloreductase |
| IREB2 | iron responsive element binding protein 2 |
| STEAP4 | STEAP family member 4 |
| ISCA2 | iron-sulfur cluster assembly 2 |
| SFXN2 | sideroflexin 2 |
| HFE2 | hemochromatosis type 2 (juvenile) |
| SFXN1 | sideroflexin 1 |
| SFXN5 | sideroflexin 5 |
| SCARA5 | scavenger receptor class A, member 5 |
| APEX2 | APEX nuclease (apurinic/apyrimidinic endonuclease) 2 |
| HPX | Hemopexin |
| HIF1AN | hypoxia inducible factor 1, alpha subunit inhibitor |

Significance analysis of microarray analysis was applied to the 63 selected probe sets in the different samples with 1000 permutations, a fold change of two and a false discovery rate of 0% (t-test).

Gene expression microarray data from four independent cohorts of patients diagnosed with MM were used. Affymetrix gene expression data are publicly available via the online Gene Expression Omnibus (http://www.ncbi.nlm.nih.gov/qeo/) under accession number GSE2658 and GSE9782. They were performed using Affymetrix HG-U133 plus 2.0 microarrays for the two cohorts of patients. The data were analyzed with Microarray Suite version 5.0 (MAS 5.0), using Affymetrix default analysis settings and global scaling as normalization method. The trimmed mean target intensity of each array was arbitrarily set to 500.

In each cohort, the statistical significance of overall survival (OS) of the expression of each probe set of the iron list was calculated by the log-rank test. Multivariate analysis was performed using the Cox proportional hazards model. Survival curves were plotted using the Kaplan-Meier method in the platform Genomicscape (Kassambara et al., 2015). Probe sets with a common prognosis value in the two cohorts were selected. To gather their prognostic information within one parameter, the Iron Score of MM was built as the sum of the beta coefficients weighted by ±1 according to the patient signal above or below the probe set Maxstat value (Kassambara et al., 2012).

### Human Myeloma Cell Lines (HMCLs)

The OPM2 cell line was purchased from the DSMZ (Leibniz-Institut DSMZ—Deutsche Sammlung von Mikroorganismen und Zellkulturen GmbH, Germany). XG-1 and XG-7 were obtained as previously described (Moreaux et al. Haematologica 2011).

They were maintained according to the supplier's recommendations. Cultures were maintained at 37° C. in a humidified atmosphere with 5% CO2.

### Reagents

Deferasirox (from Selleckchem S1712) was dissolved in dimethyl sulfoxide (DMSO) to a concentration of 50 mM respectively. Ironomycin also named ‘AM5’ in the patent application WO2016/038223 and AM-23 was dissolved in dimethyl sulfoxide (DMSO) to a concentration of 10 mM. Melphalan (Aspen, 16.4 mM), Lenalidomide (6305, Tocris, 100 mM), Pomalidomide (S1567, Selleckchem, 10 mM), carfilzomib (from Selleckchem S2853, 50 mM), AZD-5991 (from Selleckchem, S8643, 10 mM).

### Cell Viability Assay

HMCL cell lines were cultured for 4 days in 96-well flat-bottom microtiter plates in RPMI 1640 medium or DMEM medium, 10% or 20% FCS (control medium) in the presence of various compounds. The number of viable cells in culture was determined using the Cell Titer-Glo Luminescent Cell Viability Assay from Promega, Madison, WI, USA using a Centro LB 960 luminometer (Berthold Technologies, Bad Wildbad, Germany).

This test is based on quantitation of the intracellular ATP present, which signals the presence of metabolically active cells. Data are expressed as the mean percentage of six replicates, normalized to the untreated control.

### Flow Cytometry Analyses

Annexin V-PE staining for apoptosis analysis was performed using the “PE Annexin V Apoptosis Detection Kit I” (559763, Becton Dickinson).

The cell cycle progression was studied by flow cytometry using the Apoptosis, DNA Damage, and Cell Proliferation Kit (562253, Becton Dickinson). Briefly, cells were labeled with bromodeoxyuridine (BrdU), an analog of the DNA precursor thymidine that can be incorporated into newly synthesized DNA and detected with an antibody against BrdU to measure cell proliferation. After this labeling, the cells were fixed, permeabilized and treated with DNase to expose the BrdU epitopes. Following this treatment, cells were simultaneously stained with fluorochrome-labeled anti-BrdU, anti-cleaved Poly A DP-ribose polymerase 1 (PARP), anti-H2AX phosphorylated at serine 139. They were also stained with DAPI to determine DNA content. Finally, cells were resuspended in staining buffer and analyzed by flow cytometry (Fortessa, Becton Dickinson).

### Primary MM Cells

Bone marrow samples were collected after patients' written informed consent in accordance with the Declaration of Helsinki and institutional research board approval from Montpellier University hospital. Cells are obtained from lymph nodes or blood of 5 patients with MM. Cells are obtained by density gradient separation and qualified by Flow cytometry.

Cells are cultured in Gibco® RPMI-1640 (Glutamax) medium (#6187-010) with 10% FBS at a density of 0.5×106 Cell/mL with 2 ng/mL of interleukin-6. Cells are seeded 24H after thawing and treated with various compounds during 72H.

Total cells were counted with trypan blue and stained with the panel CD38-APC, CD138-PE (Beckman coulter) and analyzed by flow cytometry (Fortessa cytometer, BD Pharmigen). Tumorous MM cells were gated on CD38+ and CD138+.

Western-blot:

The total cell lysates were obtained with RIPA 1× lysis buffer (#9806, Cell Signaling®) according with the supplier recommendations.

Protein lysates migrate on 10% polyacrylamide gel (NP-0301, Novex, Life Technologies®), in MOPS 1× running buffer (NP0001, Novex, Life Technologies®) or MES 1× running buffer NP-0002, Novex, Life Technologies®) and proteins are transferred to nitrocellulose membrane (1B301001, I-Blot Transfert Starck, NuPAGE, Life Technologies®). The primary antibodies mouse-anti-phospho-Histone H2A.X (Ser139) clone JBW301 ( 1/1000, Merck Millipore), anti-H3K36me3 (#ab9050, abcam®), anti-H3K27me3 (#9733, Cell Signaling®), anti-MMSET (#65127, Cell Signaling® et ab75359, abcam), anti-MYC (#5605, Cell Signaling®) were incubated in TBS-Tween 20 0.1% (Tris-Buffered Saline, pH 7.4) with 5% non-fat milk or Bovine serum albumin (Sigma-Aldrich, A7906). Protein levels are objectified by labeling with an anti-α-tubulin mouse monoclonal antibody (Sigma, T9026, St Louis, MO, USA 1/1000) or anti-Histone 3 (ab18521, abcam). Primary antibodies are visualized with secondary anti-rabbit antibodies (Sigma®, A9169) or anti-mouse antibodies (Jackson, 115-036-068) coupled to peroxidase allows the development by chemiluminescence by Western Lightning ECL (NEL121001EA, Perkin Elmer®). Quantification of protein levels was performed with Image J® software (National Institutes of Health, Bethesda, MD, USA).

### Quantification of the Interaction Effect

The interaction between the drugs tested in vitro was investigated with a concentration matrix test, in which increasing concentration of each single drug were assessed with all possible combinations of the other drugs. For each combination, the percentage of expected growing cells in the case of effect independence was calculated according to the Bliss equation (Combes et al., 2019):

fuC=fuA·fuB

where fuC is the expected fraction of cells unaffected by the drug combination in the case of effect independence, and fuA and fuB are the fractions of cells unaffected by treatment A and B, respectively. The difference between the fraction of living cells in the cytotoxicity test and the fuC value was considered as an estimation of the interaction effect, with positive values indicating synergism and negative values antagonism. Synergy matrix was built with the R package “SynergyFinder”.

### Measurement of Intracellular Iron (II)

Intracellular Fe2+ were measured in live cells with Biotracker 575 Red Fe2+ probe (RhoNox-1). Briefly, bone marrow cells from MM patients (n=7) were washed twice with Hanks balanced salt solution and stained with 5 μM of Biotracker 575 Red Fe2+ Dye (#SCTO30, Merck) (stock solution: 1 mM in DMSO), and incubated 1 hour at 37° C. in an incubator. Intracellular iron level was investigated by flow Cytometry in bone marrow normal B cells, normal plasma cells and malignant plasma cells using LSRFortessa cytometer (BD Bioscience).

### Results

### Prognostic Genes of MM

Maxstat R function showed that 6 genes out of the 63 investigated have a prognostic value in two independent cohorts of MM patients treated by High dose therapy and autologous stem cell transplantation (TT2 cohort, n=345 and HM cohort, n=206) as disclosed in FIG. 2 and Table 4 hereunder.

TABLE 4

| ID Probe set | | | | | | | | |
|---|---|---|---|---|---|---|---|---|
| (Affymetrix | | | | | | | | |
| HG-U133 | | | | | | | | |
| plus 2.0 | | | | | Muktiple | Hazard_Ratio | | |
| microarrays) | Name | Maxstat_Cutpoint | Chisq | pvalue | testing | (HR) | expB | Prognostic |
| 222453_at | CYBRD1 | 11 | 9.8 | 0.0018 | 1.13E−02 | 0.55 | −0.2596373 | Good |
| 200878_at | EPAS1 | 108 | 22 | 2.70E−06 | 1.70E−04 | 0.37 | −0.4317983 | Good |
| 209004_s_at | FBXL5 | 3611 | 10 | 0.0016 | 1.12E−02 | 0.55 | −0.2596373 | Good |
| 204788_s_at | PPOX | 990 | 15 | 0.00012 | 2.52E−03 | 2.1 | 0.32221929 | Bad |
| 212110_at | SLC39A14 | 4060 | 13 | 0.00026 | 2.73E−03 | 0.47 | −0.3279021 | Good |
| 205542_at | STEAP1 | 325 | 6.1 | 0.013 | 4.55E−02 | 1.7 | 0.23044892 | Bad |

High expression of three genes was associated with a good prognosis including CYBRD1, EPAS1 and SLC39A14. At the opposite, high expression of two genes was associated with a poor prognosis: STEAP1 and PPOX genes.

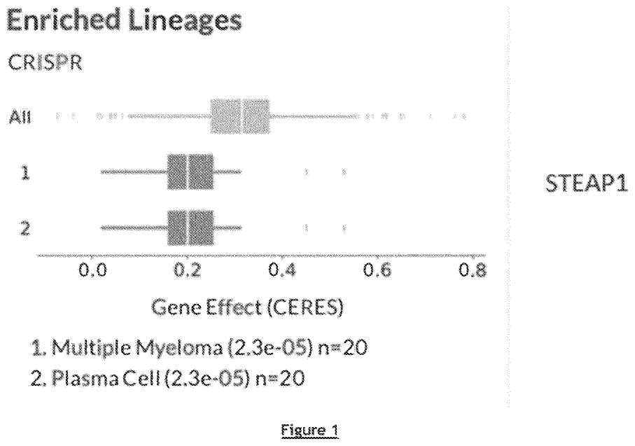

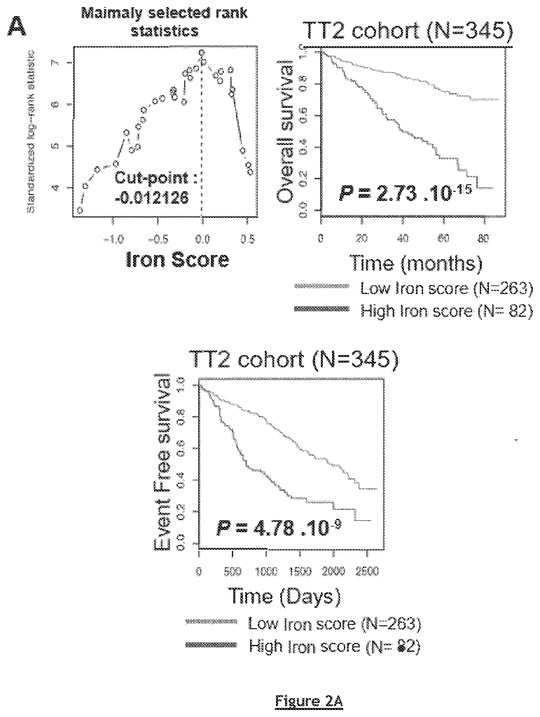

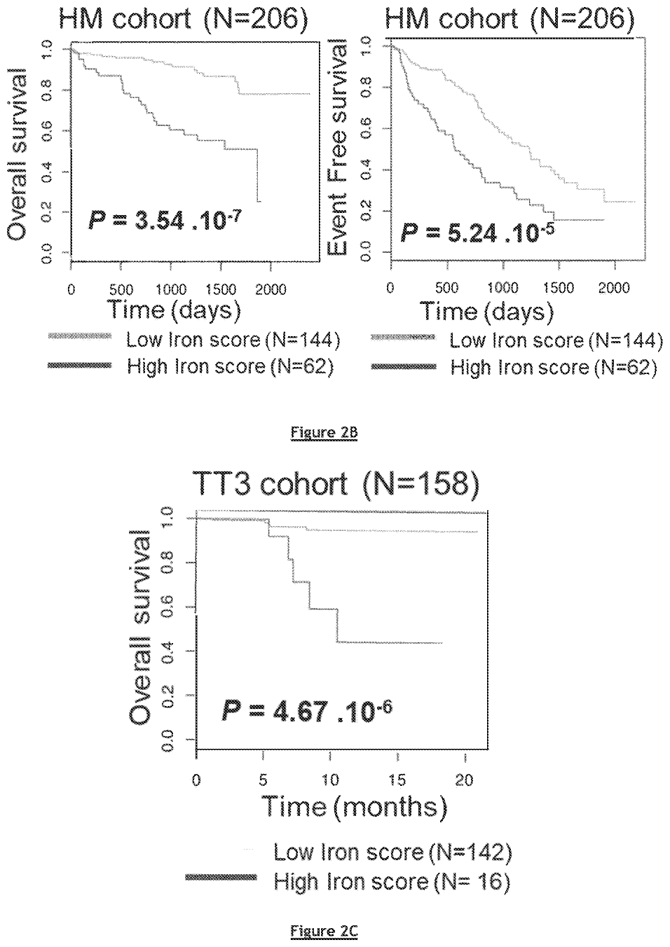

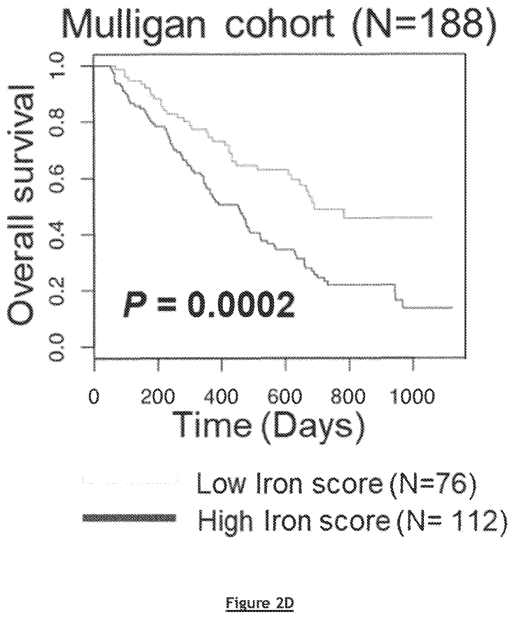

Among these genes, STEAP1 is overexpressed in several cancers in association with a poor outcome (Moreaux et al. BBRC 2012). Interestingly, analyzing CRISPR-Cas9 screening in 20 MM cell lines (Dependency Map data of the Broad Institute, www.depmap.org) revealed STEAP1 as a significant essential myeloma gene (P=2.3E-5, FIG. 1).

Based on these prognostic genes, we created a gene expression profile (GEP)-based risk score as the sum of the beta coefficients of the Cox model for each prognostic gene weighted by ±1 according to the patient signal above or below the probe set Maxstat value as previously reported (Herviou et al., 2018). Patients were ranked according to increased prognostic score and for a given score value X, the difference in survival of patients with a prognostic score ≤X or >X was computed using Maxstat analysis (Moreaux et al. MCT 2012; BJC 2013).

Maxstat algorithm segregated the TT2 cohort into two groups with 23.8% of the patients with an iron score >−0.012126 and 73.2% of the patients with an iron score ≤−0.012126 with a maximum difference in overall survival (OS; FIG. 2A). Patients with high-risk iron score have a median OS of around 40 months versus not reached for patients with low iron score (P=2.73 0.10⁻¹⁵)) in the TT2 cohort (FIG. 2A). The prognostic value of the iron score was validated in the three additional independent cohorts for OS (FIGS. 2B, C and D)

So the iron score was significantly associated with high-risk MM in 4 independent cohorts of patients (FIG. 2). These data demonstrated that high iron score allow to identify MM patient with a poor outcome and dysregulation of iron metabolism that could benefit from targeted therapy.

### Effect of Ironomycin (AM5) and AM23 on MM Cell Lines

The therapeutic interest of iron metabolism inhibitors AM5 (Ironomycin) and AM23 was further investigated using large collection of 18 MM cell lines (Moreaux J et al. Haematologica 2011; Vikova V et al. Theranostics 2019). The IC50 of Ironomycin and AM23 respectively are illustrated in the Table 5 hereunder:

TABLE 5

| HMCL | IC₅₀ Ironomycin (nM) | IC₅₀ AM23 (nM) |
|---|---|---|
| OPM-2 | 36 | 1.4 |
| XG-1 | 239 | 21.1 |
| XG-7 | 52 | 11.8 |
| XG-19 | 27 | |
| AMO-1 | 57 | |
| XG20 | 444 | 154 |
| XG6 | 26 | 3.8 |
| RPMI | 73 | |
| XG2 | 24 | |
| JJN3 | 84 | |
| L363 | 13 | |
| XG11 | 51 | |
| XG24 | 19 | 8.8 |
| XG12 | 30 | 0.8 |
| XG21 | 156 | 34.3 |
| XG28 | 31 | 13 |
| MOLP-8 | 64.7 | 13 |

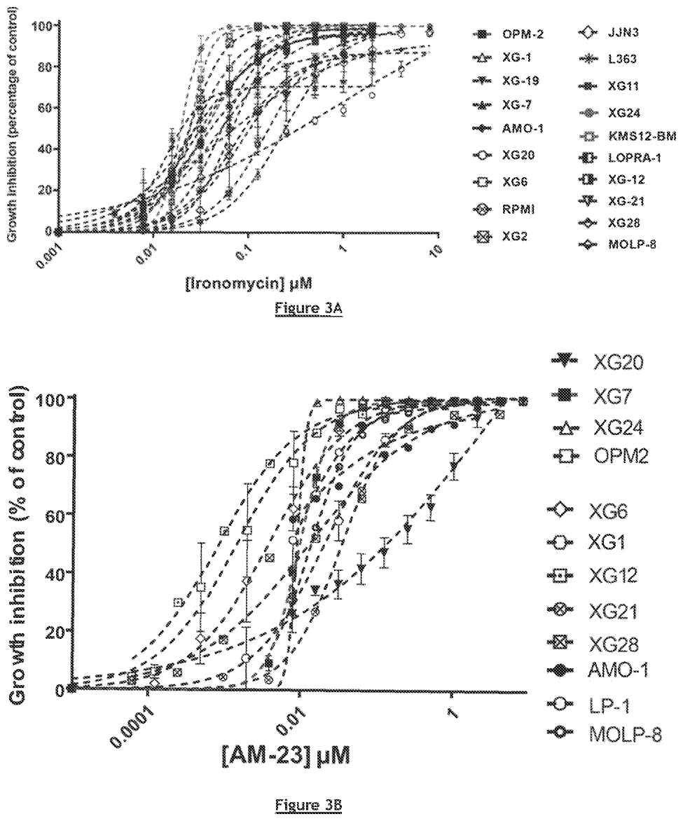

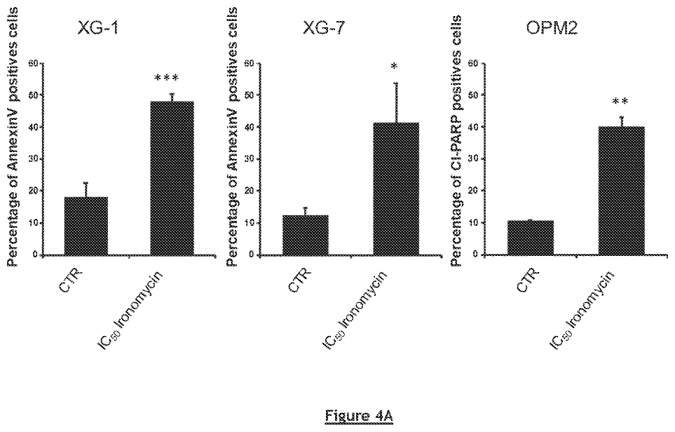

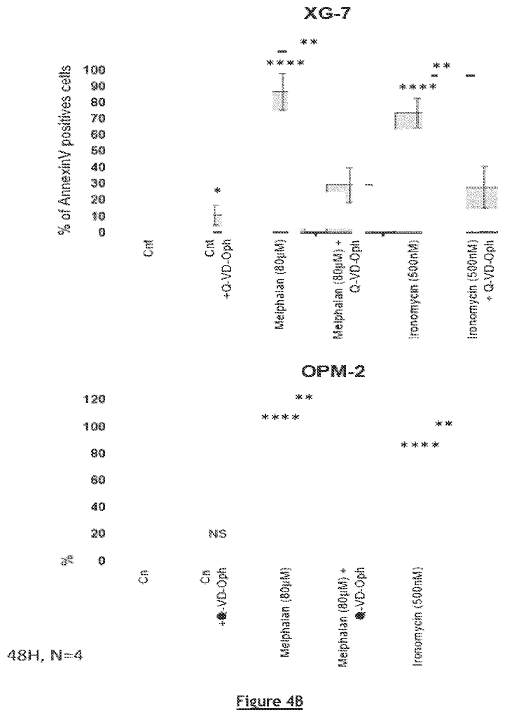

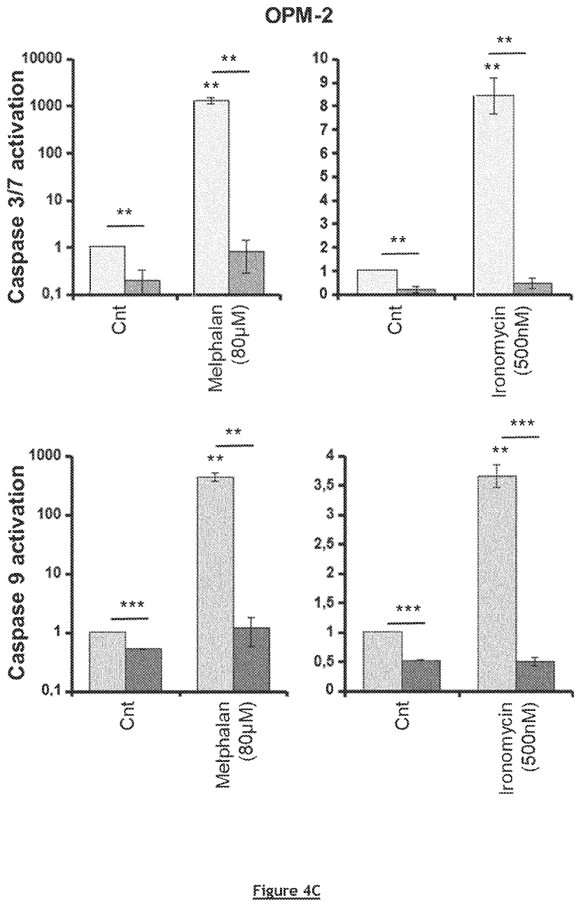

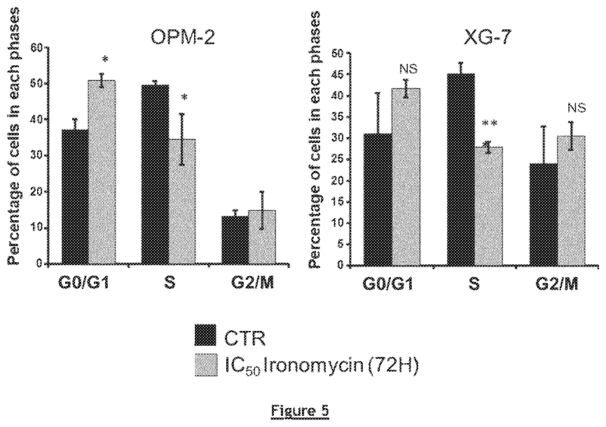

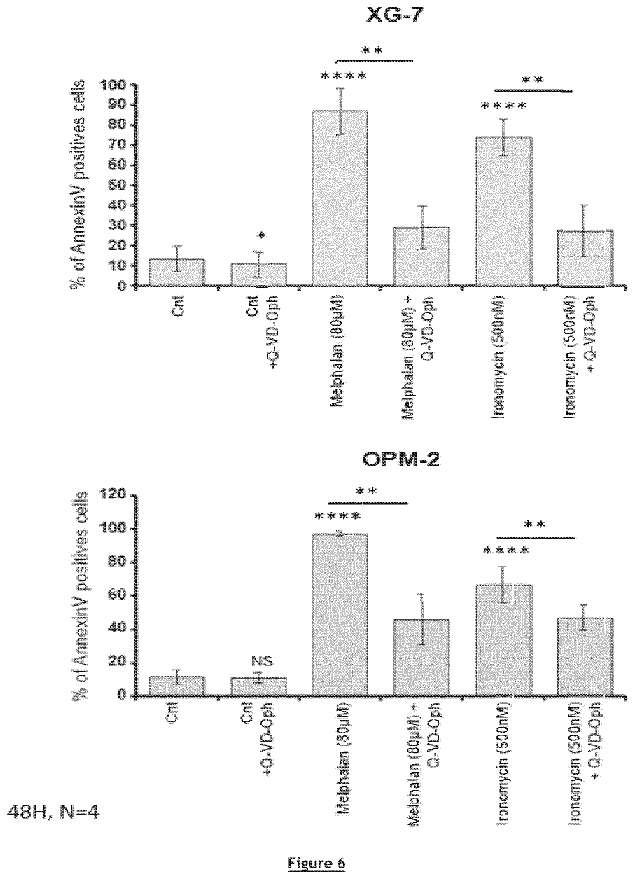

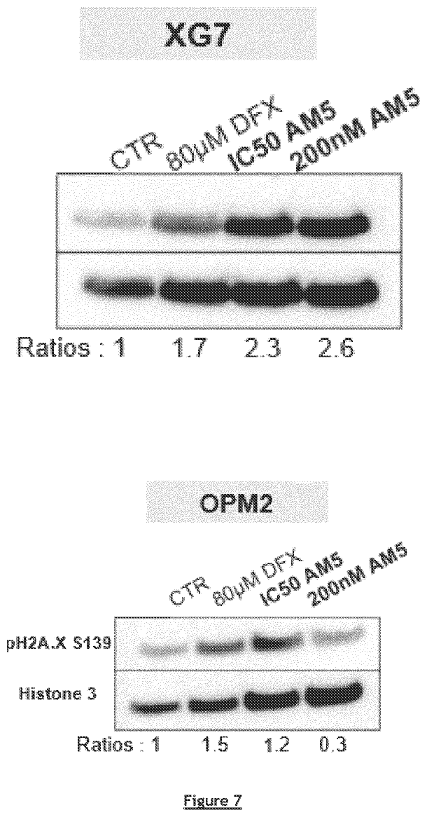

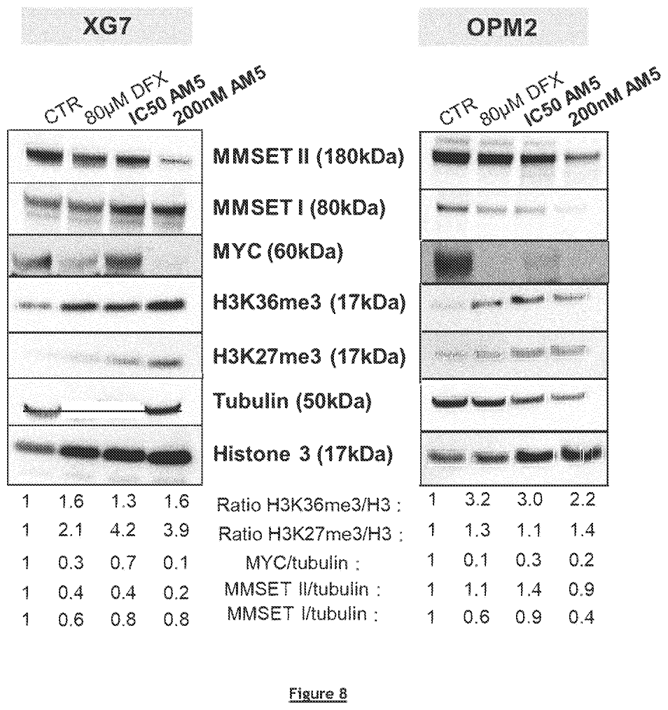

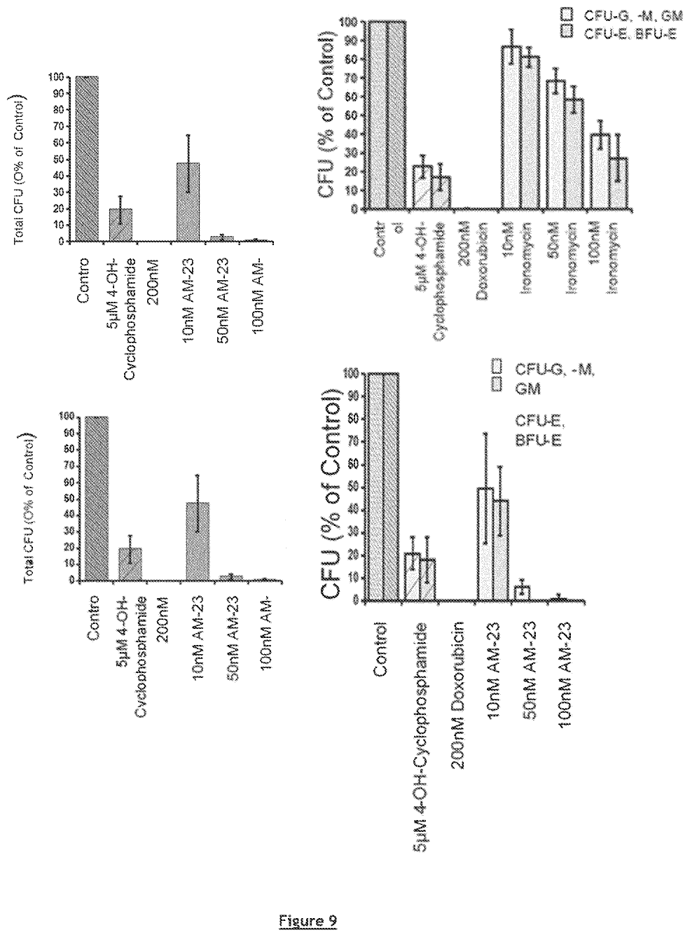

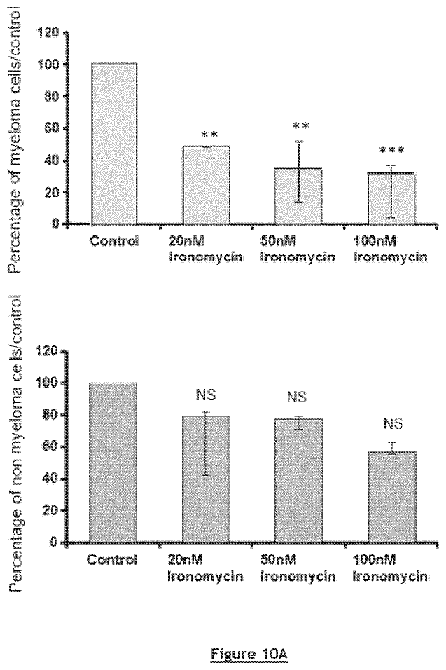

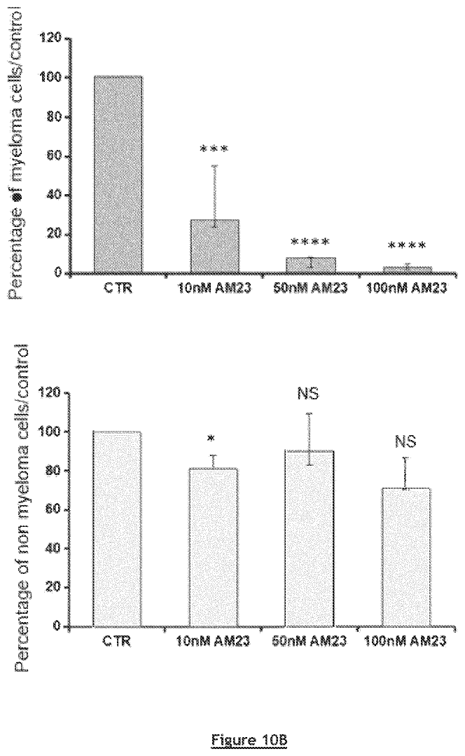

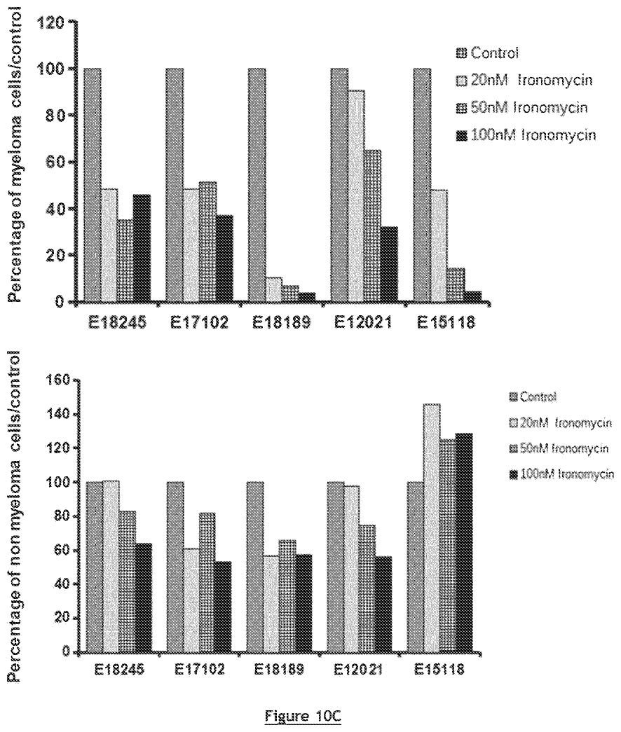

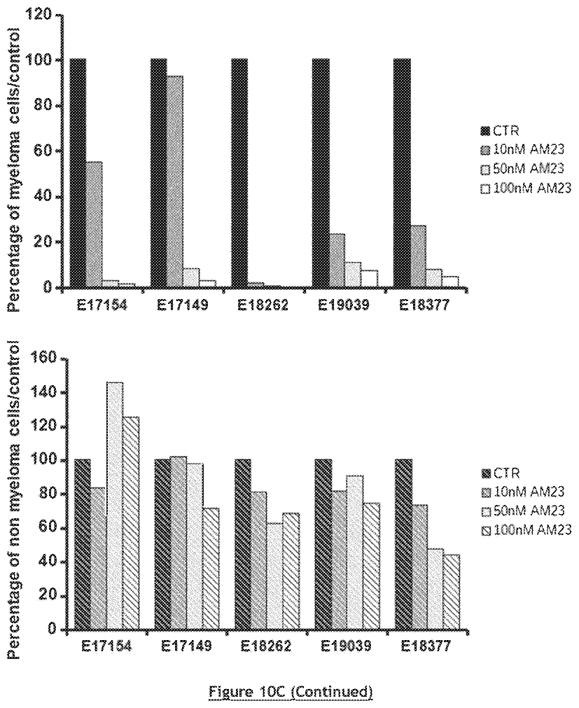

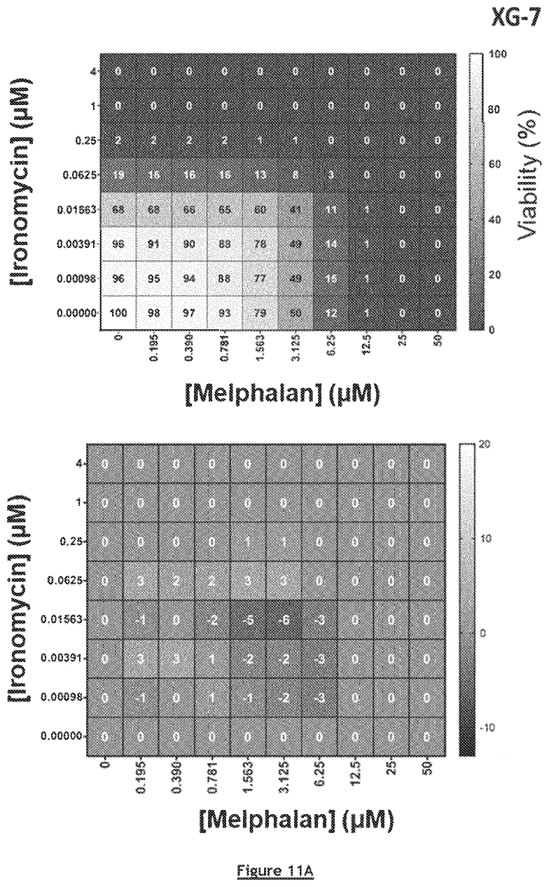

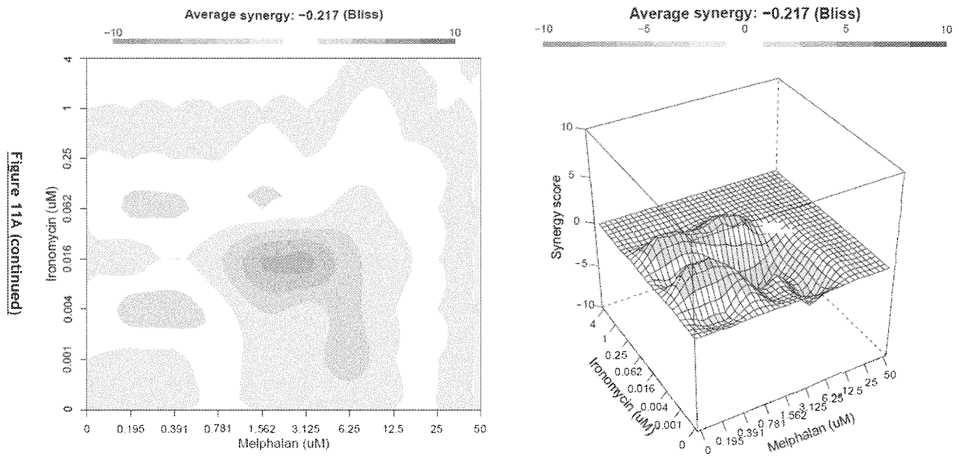

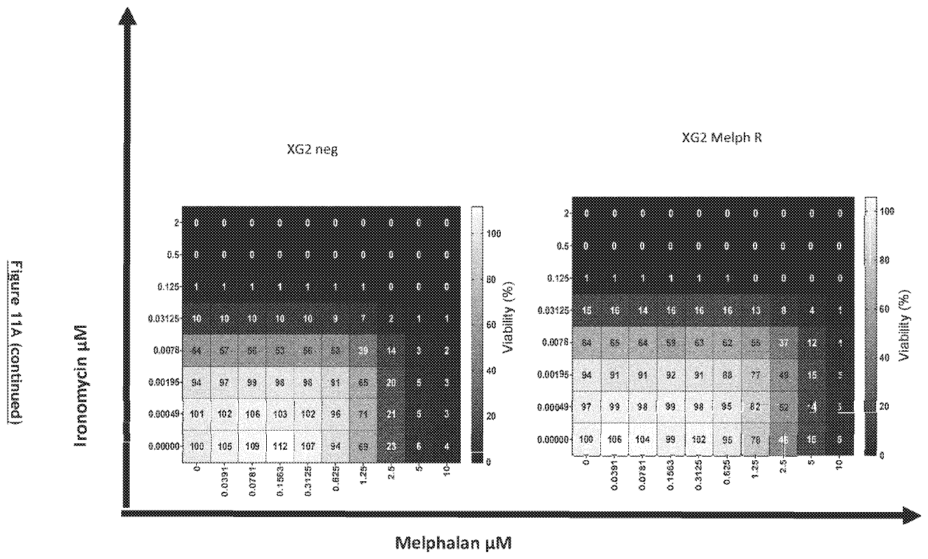

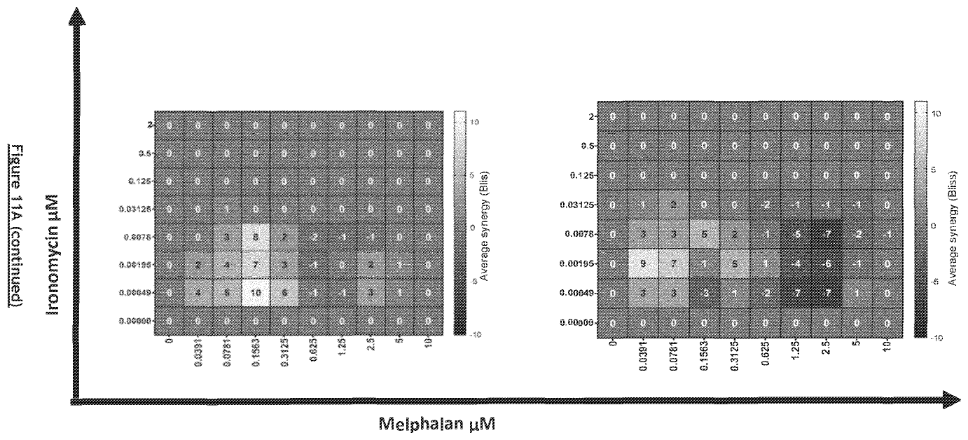

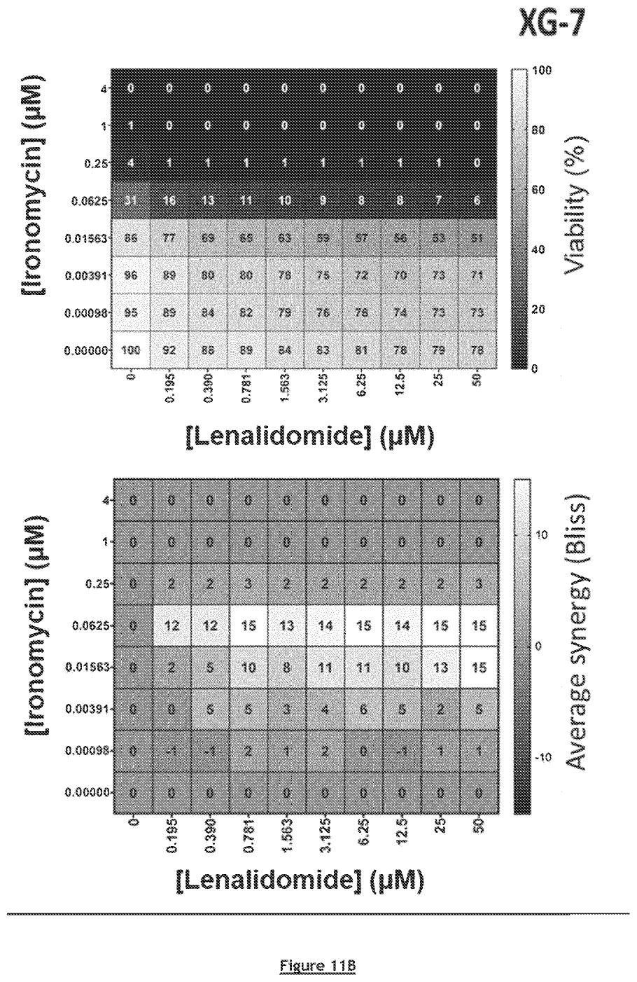

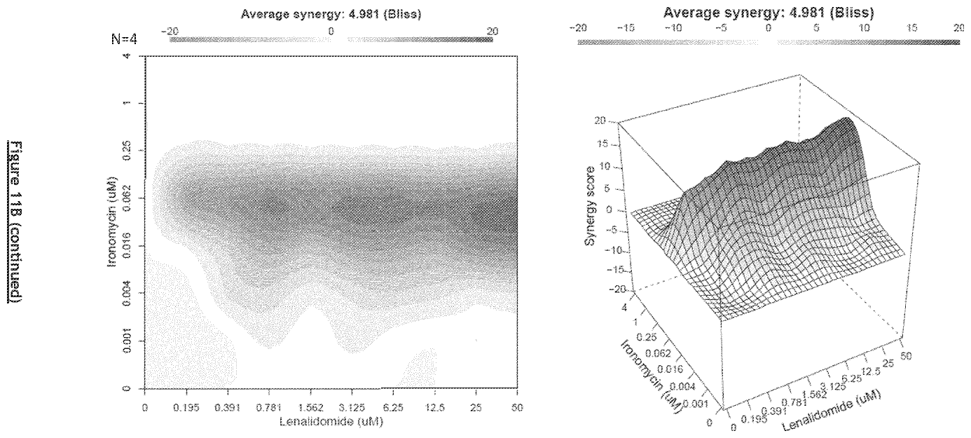

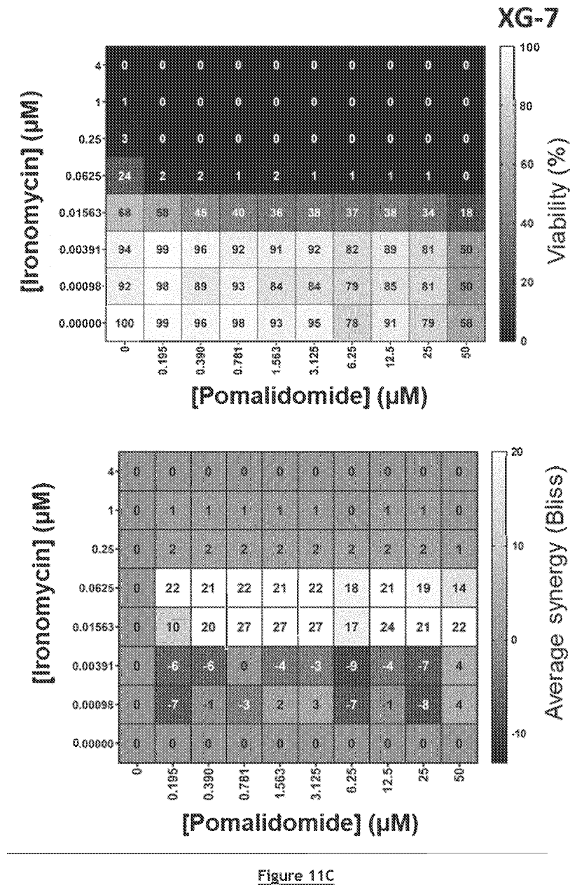

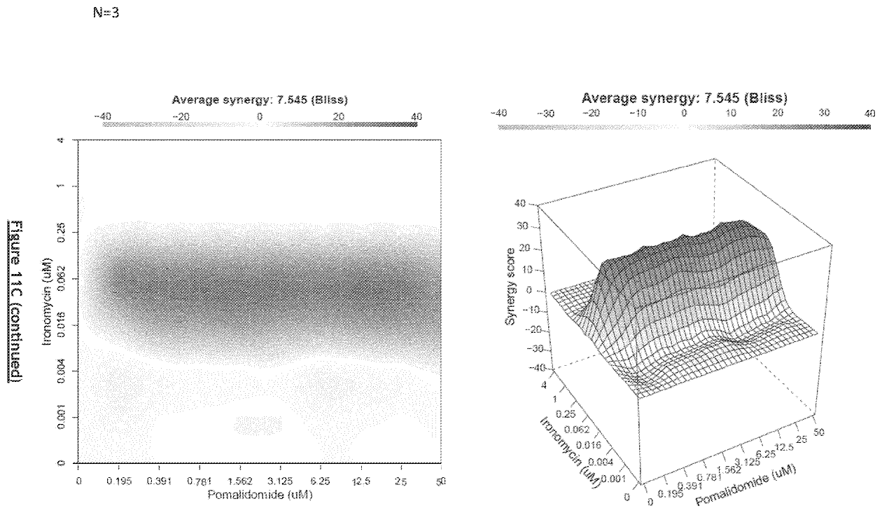

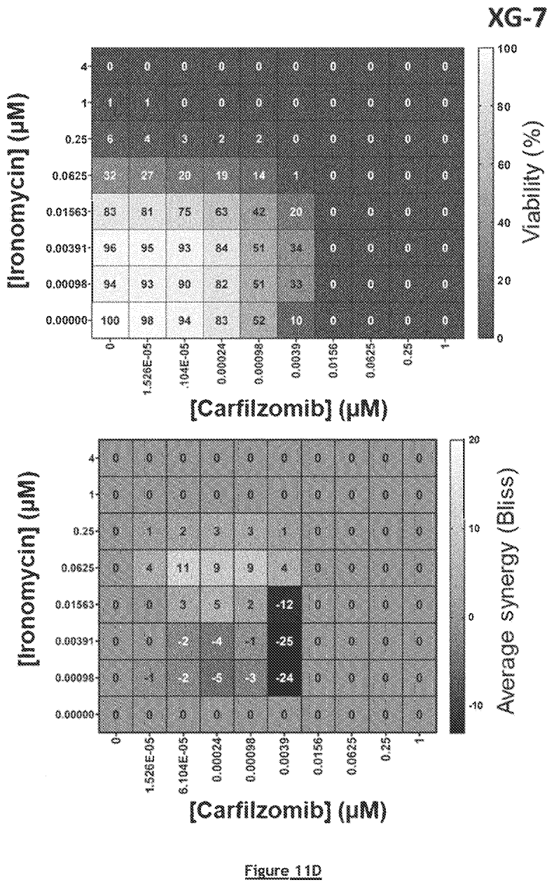

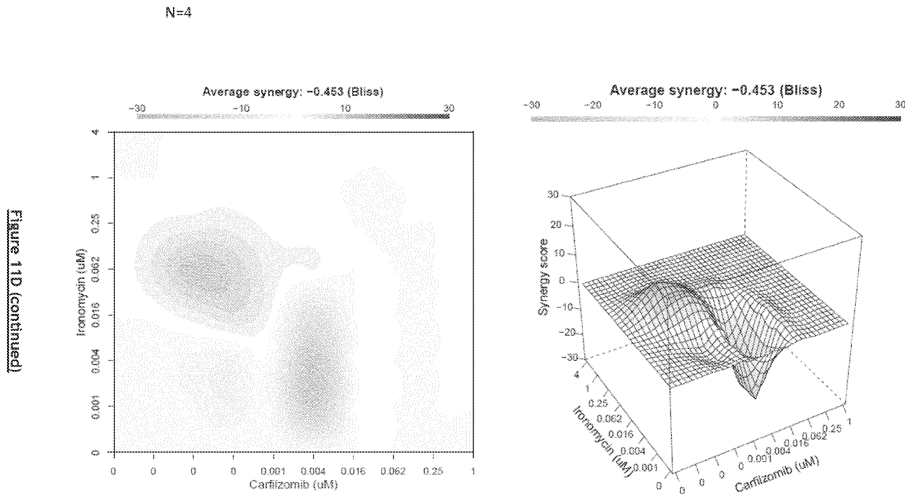

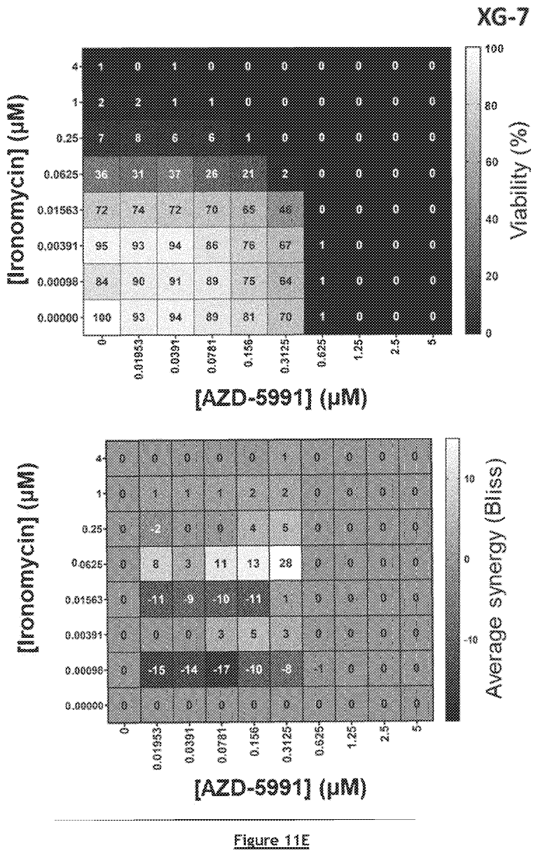

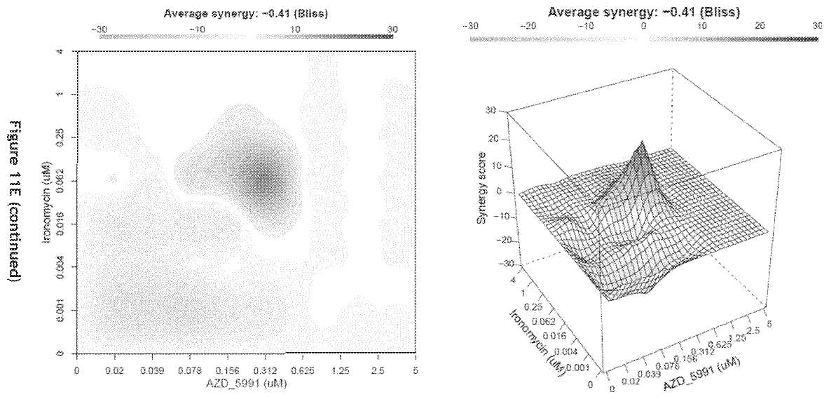

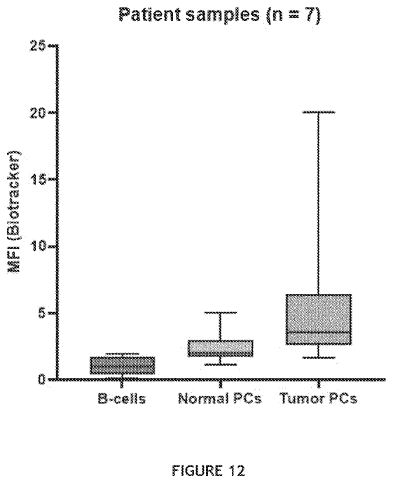

Ironomycin induces significant cell growth inhibition (FIG. 3A) in a large panel of 18 MM cell lines. We also validated the toxicity of AM23 on a panel of 12 different MM cells lines. Ironomycin treatment induces apoptosis (FIG. 4A), inhibition of proliferation of MM cells (FIG. 5), and DNA double strand breaks (FIG. 7). Furthermore, apoptosis induced by Ironomycin was not reversed by Iron supplementation (FIG. 6). Apoptosis induced by ironomycin is associated with activation of caspases 3/7 and 9 and could be partially inhibited by pan caspase inhibitor Q-VD-Oph (FIGS. 4B and C).

Furthermore, we identified that Ironomycin downregulates the expression of MMSET in MM cell lines characterized by t(4; 14) translocation. Since t(4; 14) translocation is associated with an adverse outcome in MM, Ironomycin could be of therapeutic interest in this subgroup of patients. We also identified a significant downregulation MYC protein expression after treatment by Ironomycin. MYC is a major oncogene in MM. A significant deregulation of H3K36 methylation following treatment by Ironomycin was also identified. Altogether, these data indicate that Ironomycin affects major oncogenes involved in MM biology and deregulated epigenetic profiles in MM cells.

With major importance, we validated the therapeutic interest of Ironomycin and AM23 on primary MM cells of patients (n=5) without significant toxicity for non-tumoral cells (FIG. 8). Regarding the toxicity of Ironomycin and AM23 on normal hematopoietic progenitors, Ironomycin induces low toxicity normal hematopoietic progenitors compared to AM23 (FIG. 9).

### Combination of Ironomycin with Conventional Chemotherapy Used in MM

Combination of Ironomycin with conventional chemotherapy used in MM was further tested. Interestingly, we identified a synergistic effect when Ironomycin is combined with melphalan, an alkylating agent used in the treatment of MM (FIG. 10).

Furthermore, the combination remains synergistic in melphalan resistant cell line (XG2 MeIR) developed in our laboratory as disclosed in de Boussac et al (2019) (FIG. 11A). These underlined a therapeutic interest to combine Ironomycin with melphalan and revealed that Ironomycin could be interesting to overcome melphalan resistance in MM. We also identified a synergy when Ironomycin is combined with immunomodulatory agents including Lenalidomide and Pomalidomide (FIGS. 11B and C).

When Ironomycin is combined with carfilzomib or AZD5991 (a MCL-1 inhibitor under clinical study), an additive effect was identified (FIGS. 11D and E).

### Measurement of Intracellular Fe2+ in Malignant MM Cells Compared to Normal B and Normal Plasma Cells

We investigated the level of intracellular iron in MM samples using RhoNox-1 probe. Of interest, and as illustrated in FIG. 12, MM cells of patients demonstrate a significant higher level of intracellular iron (p<0.05) compared with normal plasma cells or normal B cells from the bone marrow microenvironment supporting the observed high sensitivity of MM cells to ironomycin.

Altogether, these data demonstrated that a subgroup of high-risk MM patients could be identified with the iron-score and could benefit from Iron metabolism inhibitors and especially Ironomycin or AM23. Furthermore, combination of Ironomycin with melphalan and immunomodulatory agents demonstrated a synergistic effect.

### REFERENCES

Barlogie B, Tricot G, Rasmussen E, Anaissie E, van Rhee F, Zangari M et al. Total therapy 2 without thalidomide in comparison with total therapy 1: role of intensified induction and posttransplantation consolidation therapies. Blood 2006; 107: 2633-2638.
Combes, E., Andrade, A. F., Tosi, D., Michaud, H.-A., Coquel, F., Garambois, V., Desigaud, D., Jarlier, M., Coquelle, A., Pasero, P., et al. (2019). Inhibition of Ataxia-Telangiectasia Mutated and RAD3-related (ATR) overcomes oxaliplatin resistance and promotes anti-tumor immunity in colorectal cancer. Cancer Res.
de Boussac H, Bruyer A, Jourdan M, et al. Kinome expression profiling to target new therapeutic avenues in multiple myeloma. Haematologica. 2019.
Herviou, L., Kassambara, A., Boireau, S., Robert, N., Requirand, G., Müller-Tidow, C., Vincent, L., Seckinger, A., Goldschmidt, H., Cartron, G., et al. (2018). PRC2 targeting is a therapeutic strategy for EZ score defined high-risk multiple myeloma patients and overcome resistance to IMiDs. Clin. Epigenetics 10, 121.
Hose, Dirk et al. “Proliferation is a central independent prognostic factor and target for personalized and risk-adapted treatment in multiple myeloma.” Haematologica vol. 96, 1 (2011): 87-95. doi:10.3324/haematol.2010.030296.
Kassambara, A., Hose, D., Moreaux, J., Walker, B. A., Protopopov, A., Reme, T., Pellestor, F., Pantesco, V., Jauch, A., Morgan, G., et al. (2012). Genes with a spike expression are clustered in chromosome (sub)bands and spike (sub)bands have a powerful prognostic value in patients with multiple myeloma. Haematologica 97, 622-630.
Kassambara, A., Reme, T., Jourdan, M., Fest, T., Hose, D., Tarte, K., and Klein, B. (2015). GenomicScape: an easy-to-use web tool for gene expression data analysis. Application to investigate the molecular events in the differentiation of B cells into plasma cells. PLoS Comput. Biol. 11, e1004077.
Knight R. IMiDs: a novel class of immunomodulators. Semin Oncol. 2005 August; 32(4 Suppl 5):S24-30.
Miller, L. D., Coffman, L. G., Chou, J. W., Black, M. A., Bergh, J., D'Agostino, R., Torti, S. V., and Torti, F. M. (2011). An iron regulatory gene signature predicts outcome in breast cancer. Cancer Res. 71, 6728-6737.
Moreaux et al. A high-risk signature for patients with multiple myeloma established from the molecular classification of human myeloma cell lines. Haematologica. 2011 April; 96(4):574-82.
Moreaux J, Kassambara A, Hose D, Klein B. STEAP1 is overexpressed in cancers: A promising therapeutic target. Biochem Biophys Res Commun. 2012; 429(3-4):148-155.
Moreaux J, Reme T, Leonard W, et al. Development of gene expression-based score to predict sensitivity of multiple myeloma cells to DNA methylation inhibitors. Mol Cancer Ther. 2012; 11(12):2685-2692.
Moreaux J, Reme T, Leonard W, et al. Gene expression-based prediction of myeloma cell sensitivity to histone deacetylase inhibitors. Br J Cancer. 2013; 109(3):676-685.
Mulligan cohort: Mulligan, G. et al. Gene expression profiling and correlation with outcome in clinical trials of the proteasome inhibitor bortezomib. Blood 109, 3177-3188 (2007).
Pineda-Roman M, Zangari M, Haessler J, Anaissie E, Tricot G, van Rhee F et al. Sustained complete remissions in multiple myeloma linked to bortezomib in total therapy 3: comparison with total therapy 2. Br J Haematol 2008; 140: 625-634.
Vikova V et al. Comprehensive characterization of the mutational landscape in multiple myeloma cell lines reveals potential drivers and pathways associated with tumor progression and drug resistance. Theranostics 2019; 9(2):540-553.
Yamamoto et al. Harnessing the Immune System Against Multiple Myeloma: Challenges and Opportunities. Frontiers in Oncology, January 2021, Volume 10, Article 606368 (2021).

1. A compound of formula (I), enantiomers, mixture of enantiomers, diastereoisomers and mixture of diastereoisomers thereof: 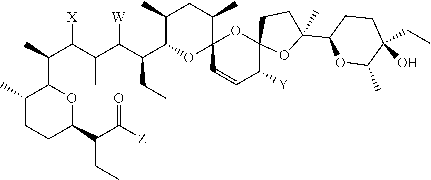
wherein:
—W is selected from the group consisting of ═O; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈;
—X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
—Y is selected from the group consisting of —OH; —N—OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
R₃ is selected from the group consisting of H; (C₁-C₆)-alkyl; (C₁-C₆)-alkyl-aryl;
R₄ and R₅, identical or different, are selected from the group consisting of H; (C₁-C₆)-alkyl; aryl and (C₁-C₆)-alkyl-aryl;
R₆, R₇ and R₈, identical or different, are selected from the group consisting of (C₁-C₆)-alkyl; aryl and (C₁-C₆)-alkyl-aryl;
—Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
R₉ and R₁₀, identical or different, are selected from the group consisting of H, (C₁-C₆)-alkyl, aryl and (C₁-C₆)-alkyl-aryl;
R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl n=0, 2, 3, 4, 5 or 6,
with the proviso that at least one of W, X and Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
for use in the treatment of Multiple Myeloma (MM).

2. A compound of formula (I) for use according to claim 1, wherein X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, (C₃-C₁₆)-cycloalkyl, (C₁-C₆)-alkyl-aryl and (C₁-C₆)-alkyl-heteroaryl.

3. A compound of formula (I) for use according to claim 1, wherein X is OH, Z is OH and Y is NR₁R₂ where R₁ is H and R₂ is selected from the group consisting of (C₈-C₁₄)-alkyl; (C₃-C₅)-alkenyl; (C₃-C₅)-alkynyl, (C₃-C₆)-cycloalkyl, benzyl, and CH₂-pyridynyl.

4. A compound of formula (I) for use according to claim 1, wherein W is ═O, X is OH, Z is OH, and Y is NR₁R₂ where R₁ is H and R₂ is a (C₃-C₅)-alkynyl group.

5. A compound of formula (I) for use according to claim 1, wherein W is ═O, X is OH, Z is OH, and Y is NR₁R₂ where R₁ is H and R₂ is a (C₃-C₆)-cycloalkyl group.

6. A pharmaceutical composition comprising in a pharmaceutical acceptable vehicle, at least a compound of formula (I) according to claim 1, for use in a method for treating subjects having Multiple Myeloma (MM).

7. A pharmaceutical composition according to claim 6, wherein the compound of formula (I) is with W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl.

8. A pharmaceutical composition according to claim 6 for use in a method for treating subjects likely to display an MM relapse and/or death, or subjects refractory or resistant to a first line treatment.

9. A pharmaceutical product comprising:
(i) a compound of formula (I) according to claim 1 and
(ii) another anti-cancer agent or cellular therapy for treating MM selected in the group consisting of agents used either in chemotherapy, agents used in targeted treatment, agents used in immune therapies or combinations thereof, in particular selected in the group consisting of proteasome inhibitors (Pis), immunomodulatory agents in particular immunomodulatory drugs (IMiDs), DNA methyltransferase inhibitor, chemo drugs, inhibitors of nuclear export in particular exportin 1 inhibitor, corticosteroids, histone deacetylase (HDAC) inhibitors, therapeutic monoclonal antibodies (moAb) in particular anti-CD38, anti-SLAMF7, and/or anti BCMA, antibodies drug conjugates (ADC), bispecific T-cell engager (BiTES), MCL1 inhibitors and other BH3 mimetics, CART-T cells and combinations thereof,
as combination product for simultaneous, separate or staggered use in the treatment of MM.

10. A pharmaceutical product of claim 9, for use in a method for treating subjects likely to display an MM relapse and/or death, or subjects refractory or resistant to a first line treatment.

11. A pharmaceutical product comprising:
(i) a compound of formula (I) according to claim 1 wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl,
(ii) an anticancer agent selected in the group consisting of lenalidomide, pomalidomide (immunomodulatory agents), melphalan (chemo-drug), carfilzomib (proteasome inhibitor), AZD-5991 (MCL1 inhibitor), and combinations thereof and
(iii) optionally another anticancer agent or cellular therapy for treating MM selected in the group consisting of proteasome inhibitors (Pis), immunomodulatory agents, chemo drugs, inhibitors of nuclear export in particular exportin 1 inhibitor, corticosteroids, histone deacetylase (HDAC) inhibitors, therapeutic monoclonal antibodies (moAb) in particular anti-CD38, anti-SLAMF7, and/or anti BCMA, antibodies drug conjugates (ADC), bispecific T-cell engager (BiTES), MCL1 inhibitors and other BH3 mimetics, CART-T cells and combinations thereof.

12. A pharmaceutical product according to claim 11, wherein:
(i) the compound of formula (I) is such as W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl, and
(ii) the anticancer agent is selected in the group consisting of lenalidomide, pomalidomide, melphalan, and a combination thereof.

13. A pharmaceutical product according to claim 11, for use in a method for treating subjects likely to display an MM relapse and/or death, or subjects refractory or resistant to a first line treatment.

14. A pharmaceutical composition according to claim 6, for use in the treatment of a MM subject that has been identified as having a poor outcome by a method comprising the steps of:
a) Measuring the expression level of at least 3, in particular at least 5 genes and/or proteins encoded by the said at least 3, in particular the said at least 5 genes selected in the group consisting of CYBRD1, EPAS1, FBXL5, PPOX, SLC39A14, and STEAP1 involved in the iron metabolism, in a biological sample obtained from said subject;
b) Calculating a score value from said expression level obtained at step a)
c) Classifying and identifying the said subject as having a poor outcome according to the score value in comparison to a predetermined reference value (PRV).

15. An in vitro method for identifying MM subject with a poor outcome that may benefit from a therapeutic treatment comprising a compound of formula (I), enantiomers, mixture of enantiomers, diastereoisomers and mixture of diastereoisomers thereof as defined in claim 1, comprising the steps of:
a) Measuring the expression level of at least 3, in particular at least 5 genes and/or proteins encoded by the said at least 3, in particular the said at least 5 genes selected in the group consisting of CYBRD1, EPAS1, FBXL5, PPOX, SLC39A14, and STEAP1 involved in the iron metabolism, in a biological sample obtained from said subject;
b) Calculating a score value from said expression level obtained at step a) Classifying and identifying the said subject as having a poor outcome according to the score value in comparison to a predetermined reference value (PRV).
